(19) United States
(12) Patent Application Publication
(10) Pub. No.: US 2025/0360218 A1
(43) Pub. Date: Nov. 27, 2025

(54) MITOCHONDRIA-TARGETING CANCER THERAPEUTIC COMPOSITION

(71) Applicants: Seoul National University R&DB Foundation, Seoul (KR); Seoul National University Hospital, Seoul (KR)

(72) Inventors: Hyung-Jun Im, Seoul (KR); Wooseung Lee, Seoul (KR); Miyeon Jeon, Seoul (KR); Jeong-seob Lee, Seoul (KR); Byung Chul Lee, Gyeonggi-do (KR); Sang Hee Lee, Gyeonggi-do (KR)

(73) Assignees: Seoul National University R&DB Foundation, Seoul (KR); Seoul National University Hospital, Seoul (KR)

(21) Appl. No.: 18/841,365

(22) PCT Filed: Feb. 24, 2023
(86) PCT No.: PCT/KR2023/002686

§ 371 Date: Apr. 18, 2025

(30) Foreign Application Priority Data
Feb. 24, 2022 (KR) 10-2022-0024184

(51) Int. Cl. A61K 47/54; A61K 9/127; A61K 41/00; A61K 51/04; A61P 35/00

(52) U.S. Cl. CPC A61K 47/545; A61K 9/127; A61K 41/0057; A61K 51/0497; A61P 35/00; A61K 2121/00

(57) ABSTRACT

One aspect of the present disclosure relates to a compound including a photosensitizer and an imidazopyridine derivative, a liposome including the compound, and a photodynamic therapeutic composition containing the compound or the liposome. According to exemplary embodiments of the present invention, a photosensitizer and an imidazopyridine derivative that can specifically target TSPO are contained to enhance the targeting capability, leading to high binding affinity for biomarkers. In addition, liposomes are utilized not only to prevent unwanted effects on other organs in the body, but also to selectively target tumors, thereby enabling very safe photodynamic therapy. In addition, according to exemplary embodiments of the present invention, a photodynamic therapeutic composition can be provided that can be effectively used in cancer treatment as a substitute for existing anticancer drugs.

### TECHNICAL FIELD

Disclosed herein are a compound comprising an imidazopyridine derivative for targeting mitochondria in cancer cells, a liposome including the compound, and a composition for cancer treatment containing the compound or liposome.

Meanwhile, this application was supported by the following national research and development project.
[National research and development project that supported this invention]
[Task identification number]1711137217
[Task number]2021M2E8A1039564
[Implementing department] Ministry of Science and ICT
[Research management specialist agency] National Research Foundation of Korea
[Project Name] Research based on future innovation utilizing radiation (R&D)
[Task name] Development of radiation-induced photoimmunotherapy using antibody-europium-photosensitizer composite
[Contribution rate]1/3
[Supervision institution] Seoul National University
[Research period] May 1, 2021 to Dec. 31, 2021
[Task identification number]1711144831
[Task number]2020R1C1C1009000
[Implementing department] Ministry of Science and ICT
[Research management specialist agency] National Research Foundation of Korea
[Project Name] Individual basic research (Ministry of Science and ICT) (R&D)
[Task name] Development of targeted radionuclide-photodynamic combination cancer therapy utilizing radioluminescent liposomal nanoplatform
[Contribution rate]1/3
[Supervision institution] Seoul National University
[Research period] Mar. 1, 2021 to Feb. 28, 2022
[Task identification number]1711163715
[Task number]2021R1A2C2003301
[Implementing department] Ministry of Science and ICT
[Research management specialist agency] National Research Foundation of Korea
[Project Name] Individual basic research (Ministry of Science and ICT)
[Task name] Development of pH- and temperature-sensitive nanocomposite-based nanocarrier that enhance drug loading capacity, tumor selectivity, and retention of mitochondria-targeting tumor therapeutic
[Contribution rate]1/3
[Supervision institution] Bundang Seoul National University Hospital
[Research period] Mar. 1, 2022 to Feb. 28, 2023

### BACKGROUND ART

Mitochondria are eukaryotic organelles and are equipped with a porous outer membrane and a protein-rich inner membrane. Mitochondria are organelles that play an important role in cells and are involved in ATP production, cellular respiration, and cell death regulation. In particular, mitochondria in cancer cells are known to play an important role in tumor proliferation, invasion, and metastasis by enabling cancer cells to survive under harsh conditions such as low nutrient and hypoxic conditions through glycolysis and oxidative phosphorylation. Moreover, mitochondrial biogenesis is upregulated in a small number of cancer types, and mitochondrial contribution to the progression to malignant tumor increases over time with mutations in nuclear-derived noncoding tricarboxylic acid (TCA) cycle enzymes leading to the generation of carcinogenic metabolites.

As the correlation between such mitochondria and cancer cells and their malignant progression is becoming known, mitochondria-targeting treatment for tumors is receiving attention recently. In the mitochondria-targeting treatment, anticancer drugs that target mitochondria are used to decrease the energy (adenosine triphosphate, ATP) production of mitochondria in cancer cells and increase reactive oxygen species (ROS) and membrane permeability, thereby inducing the release of apoptosis-inducing factor (AIF) and ultimately inducing fatal cancer cell apoptosis in tumor cells. In addition, since mitochondrial DNA contains only essential genes without introns and thus has no DNA repair pathway when damaged, tumor treatment targeting mitochondria is expected to be highly effective.

Translocator protein 18 kDa (TSPO) is a protein located in the outer mitochondrial membrane. TSPO is particularly closely correlated with tumor proliferation, invasion, and metastasis in tumors, and its expression is known to be specifically high in various cancers (breast cancer, prostate cancer, lung cancer, testicular cancer, bladder cancer and the like), so it is a useful biomarker for mitochondria-targeting tumor diagnosis and therapeutic strategies. Therefore, diagnostic ligands that can target TSPO, such as [¹⁸F]GE-180, [¹⁸F]DPA-714, and [¹⁸F]PBR-06, have been hitherto developed, and various nonclinical/clinical diagnostic studies are being conducted. However, although development and research of diagnostic ligands targeting TSPO are actively underway, since mitochondria are essential organelles of advanced cells, there are concerns over targeting normal organs/cells when mitochondria-targeting ligands are used singly, resulting in development and preclinical studies of TSPO-targeting ligands for cancer treatment being rarely reported to date. Hence, research on selective drug delivery systems is needed to increase the targeting specificity of therapeutic TSPO ligands for cancer cells and reduce side effects on normal cells.

Since the first FDA approval of Doxil, a lipid-based nano drug utilizing an anticancer agent in a liposome, in the 1990s, lipid-based nano drug delivery platforms have been widely studied, and recently, there have been cases of COVID-19 vaccines also encapsulating mRNA in liposomes for intracellular delivery. Liposomes are phospholipid bilayers formed of phospholipids and cholesterol, similar to cell membranes, and are not only being studied as drug delivery vehicles for the delivery of therapeutic drugs for various diseases but also have high clinical accessibility due to their high biocompatibility since both lipophilic and hydrophilic drugs can be incorporated inside the phospholipid bilayer and liposomes and there are advantages such as high drug loading rate and high synthetic reproducibility. Accordingly, cancer treatment technology that targets mitochondria in cancer cells through lipid-based nano drug delivery vehicles that minimize drug exposure to normal cells and organs in the body and enable specific uptake and drug delivery to cancer cells in the tumor is expected to be a next-generation mitochondria-targeting fusion cancer therapeutic agent with highly effective therapeutic efficacy.

### SUMMARY OF INVENTION

### Technical Problem

The present inventors have conducted extensive studies on a new technology for developing a TSPO-targeting therapeutic ligand by introducing various therapeutic drugs into a position where specific targeting of TSPO is possible using an imidazopyridine derivative, and implementing a liposome composition capable of effectively delivering the same to mitochondria in cancer cells, and have thus achieved the present invention.

In an aspect, in an exemplary embodiment of the present invention, it is intended to provide a compound comprising a photosensitizer for photodynamic therapy and an imidazopyridine derivative combined with isotopes for radiodiagnosis and radiotherapy or low molecular weight drugs for chemotherapy, which can be used to treat cancer as a substitute for existing anticancer drugs.

### Solution to Problem

In an aspect, the present invention provides a compound represented by the following Chemical Formula 1, which includes a drug and an imidazopyridine derivative. Such a compound can achieve mitochondria-targeting cancer treatment.

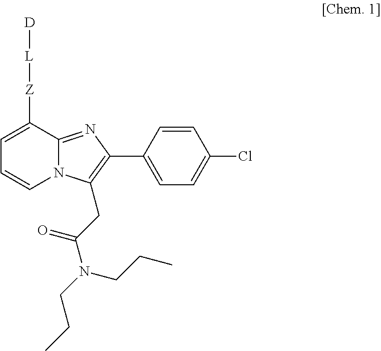

(in the formula, D is a drug; L is a linker; and Z is a bonding group (conjugation)).

Z may be selected from the group consisting of an ether, an amide, an ester, urea, urethane, thiourea, a sulfide and a disulfide. For example, Z may be

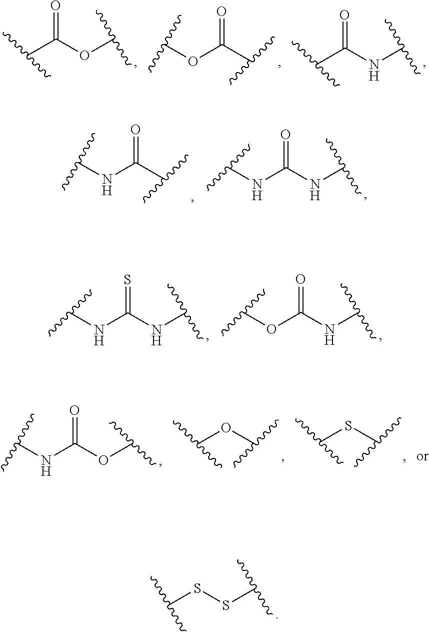

L may be -(PEG)n-, —(CH₂)n- or -phenyl-, and n may be 0 to 20.

In an aspect, the drug (D) may include a photosensitizer for the purpose of performing photodynamic therapy (PDF). For example, the photosensitizer may include one or more selected from the group consisting of porphyrin derivatives (5-aminolaevulinic acid, protoporphyrin IX, verteporfin, photofrin, benzoporphyrin, verteporfin, and phthalocyanine Pc 4), chlorin derivatives (purlytin, foscan, tetra(m-hydroxyphenyl)chlorin, bacteriochlorin, foscan, mono-aspartyl chlorin e6, and lutetium texaphyrin), phthalocyanine derivatives (zinc(III) phthalocyanine, sulfonated zinc(II), and Al(III) phthalocyanine chloride tetrasulfonic acid), merocyanine derivatives (MC540 and rhodamine complex), porphycene derivatives, heptamethine cyanine derivatives (MHI-148, IR780, IR-783, IR-786, IR-808, IR-813, R820, ICG, Pz 247, and MHI-148-783), chitosan derivatives (total phenolic compound content, sodium tripolyphosphate, and tetrasodium pyrophosphate decahydrate), methylene blue derivatives (methylene blue, dimethylene blue, new methylene blue), monoterpene derivatives (azulene), xanthene derivatives (erythrosin), toluidine blue derivatives (toluidine blue O), fluorescein derivatives, and menaquinone derivatives.

In an aspect, the drug (D) may include a metal chelator labeled with a metallic radioisotope for the purpose of performing radiodiagnosis and radiotherapy. For example, the metallic radioisotope may include one or more selected from the group consisting of ⁶⁰ Cu, ⁶¹Cu, ⁶²Cu, ⁶⁴Cu, ⁶⁷Cu, ⁶⁶Ga, ⁶⁷Ga, ⁶⁸Ga, ⁴⁴Sc, ⁴⁴SC, ⁴⁷SC, ¹¹¹In, ^{114m} In, ¹¹⁴In, ⁸⁶Y, ⁹⁰Y, ²¹²Bi, ²¹³Bi, ²¹²Pb, ²²⁵Ac, ⁸⁹Zr and ¹⁷⁷Lu, and the metal chelator may include one or more selected from the group consisting of DOTA (1,4,7,10-tetraazacyclododecane-1,4,7,10-tetraacetic acid), DOTAGA, CB-DO2A (4,10-bis(carboxymethyl)-1,4,7,10-tetraazabicyclo[5.5.2]tetradecane), TCMC (1,4,7,10-tetrakis(carbamoylmethyl)-1,4,7,10-tetraazacyclododecane), 3p-C-DEPA, p-NH2-Bn-Oxo-DO3A, TETA (1,4,8,11-tetraazacyclotetradecane-1,4,8,11-tetraacetic acid), CB-TE2A (4,11-bis-(carboxymethyl)-1,4,8,11-tetraazabicyclo[6.6.2]-hexadecane), Diamsar, NOTA (1,4,7-triazacyclononane-1,4,7-triacetic acid), p-SCN-Bn-NOTA (C-NOTA), NETA ({4-[2-(bis-carboxymethylamino)-ethyl]-7-carboxymethyl-[1,4,7]triazonan-1-yl}-acetic acid), TACN-TM (N,N′,N″, tris(2-mercaptoethyl)-1,4,7-triazacyclononane), DTPA (diethylenetriaminepentaacetic acid), CHX-A″-DTPA (2-(p-isothiocyanatobenzyl)-cyclohexyldiethylenetriaminepentaacetic acid), TRAP ((PRP9, TRAP-Pr), 1,4,7-triazacyclononane-1,4,7-tris[methyl(2-carboxyethyl)phosphinic acid]), AAZTA (1,4-bis(hydroxycarbonyl methyl)-6-[bis(hydroxylcarbonyl methyl)]amino-6-methyl perhydro-1,4-diazepine), H2dedpa (1,2-[[6-(carboxy)-pyridin-2-yl]-methylamino]ethane), H4octapa (N,N′-bis(6-carboxy-2-pyridylmethyl)-ethylenediamine-N,N′-diacetic acid), H2azapa (N,N′-[1-benzyl-1,2,3-triazole-4-yl]methyl-N,N′-[6-(carboxy)pyridin-2-yl]-1,2-diaminoethane), H5decapa (N,N″-[[6-(carboxy)pyridin-2-yl]methyl]-diethylenetriamine-N,N′,N″-triacetic acid), HBED (N,N′-bis(2-hydroxybenzyl)-ethylenediamine-N,N′-diacetic acid), SHBED (N,N′-bis(2-hydroxy-5-sulfobenzyl)-ethylenediamine-N,N′-diacetic acid), BPCA, CP256, PCTA (3,6,9,15-tetraazabicyclo[9.3.1]-pentadeca-1(15), 11,13-triene-3,6,9, -triacetic acid), DFO (desferrioxamine B), p-SCN-Bn-DFO, H6phospa (N,N′-(methylenephosphonate)-N,N′-[6-(methoxycarbonyl)pyridin-2-yl]-methyl-1,2-diaminoethane), HEHA (1,4,7,10,13,16-hexaazacyclohexadecane-N,N′,N″,N′″,N″ ″,N′″″-hexaacetic acid) and PEPA (1,4,7,10,13-pentaazacyclopentadecane-N,N′,N″,N′″,N″ ″-pentaacetic acid). The metal chelator labeled with a metallic radioisotope may be, for example, a DOTA metal chelator labeled with ⁶⁰ Cu, ⁶¹Cu, ⁶²Cu, ⁶⁴Cu, ⁶⁷Cu, ⁶⁷Ga, ⁶⁸Ga, ⁴⁴Sc, ⁴⁷Sc, ¹¹¹ In, ¹⁷⁷Lu, ⁸⁶Y, ⁹⁰Y, ²¹³Bi, ²¹²Pb or ²²⁵Ac; a CB-DO2A metal chelator labeled with ⁶⁰Cu, ⁶¹Cu, ⁶²Cu, ⁶⁴Cu, ⁶⁷Cu, ⁶⁷Ga or ⁶⁸Ga; a TCMC metal chelator labeled with ²¹²Pb; a 3p-C-DEPA metal chelator labeled with ²¹²Bi or ²¹³Bi; a TETA metal chelator labeled with ⁶⁰Cu, ⁶¹Cu, ⁶²Cu, ⁶⁴Cu or ⁶⁷Cu; a CB-TE2A metal chelator labeled with ⁶⁰Cu, ⁶¹Cu, ⁶²Cu, ⁶⁴Cu or ⁶⁷Cu; Diamsar labeled with ⁶⁰Cu, ⁶¹Cu, ⁶²Cu, ⁶⁴Cu or ⁶⁷Cu; a NOTA metal chelator labeled with ⁶⁰Cu, ⁶¹Cu, ⁶²Cu, ⁶⁴Cu, ⁶⁷Cu, ⁶⁷Ga or ⁶⁸Ga; a NETA metal chelator labeled with ¹⁷⁷Lu, ⁸⁶Y, ⁹⁰Y, ²¹²Bi or ²¹³Bi; a DTPA metal chelator labeled with ⁴⁴Sc, ⁴⁷Sc, ¹¹¹In, ¹⁷⁷Lu, ⁸⁶Y or ⁹⁰Y; a CHX-A″-DTPA metal chelator labeled with ¹¹¹In, ¹⁷⁷Lu, ⁸⁶Y, ⁹⁰Y or ²¹³Bi; a TRAP metal chelator labeled with ⁶⁷Ga or ⁶⁸Ga; an AAZTA metal chelator labeled with ⁶⁷Ga or ⁶⁸Ga; a H2dedpa metal chelator labeled with ⁶⁰Cu, ⁶¹Cu, ⁶²Cu, ⁶⁴Cu, ⁶⁷Cu, ⁶⁷Ga or ⁶⁸Ga; an H4octapa metal chelator labeled with ¹¹¹In or ¹¹⁷Lu; an H2azapa metal chelator labeled with ⁶⁰Cu, ⁶¹Cu, ⁶²Cu, ⁶⁴Cu or ⁶⁷Cu; an HBED metal chelator labeled with ⁶⁷Ga or ⁶⁸Ga; an SHBED metal chelator labeled with ⁶⁷Ga, ⁶⁸Ga, or ¹¹¹In; a BPCA metal chelator labeled with ¹¹¹In; a CP256 metal chelator labeled with ⁶⁷Ga or ⁶⁸Ga; a PCTA metal chelator labeled with ⁶⁰Cu, ⁶¹Cu, ⁶²Cu, ⁶⁴Cu, ⁶⁷Cu, ⁶⁷Ga or ⁶⁸Ga; a DFO metal chelator labeled with ⁶⁷Ga, ⁶⁸Ga or ⁸⁹Zr; or an H6phospa metal chelator labeled with ⁸⁹Zr. In this way, composites selected from the group of metallic radioisotope-labeled chelators that can be subjected to cancer diagnosis or radiotherapy through in vivo nuclear medicine examination such as positron emission tomography (PET) and single photon emission computed tomography (SPECT) may be used.

In an aspect, the drug may include a chemotherapeutic drug for the purpose of performing chemotherapy. As the chemotherapeutic drug, for example, one or more selected from the group consisting of representative anticancer drugs known as chemotherapeutic drugs, such as doxorubicin, hydroxyurea, vincristine, docetaxel, cyclophosphamide, carboplatin, methotrexate, paclitaxel, cisplatin, 5-fluorouracil, leucovorin, prednisolone, melphalan, chlorambucil, carmustine, daunorubicin, bleomycin, cytarabine, busulfan, capecitabine, 5-FU, mitomycin C, tamoxifen, bicalutamide, gonadotropin, irinotecan, belotecan, ifosfamide, temozolomide, fludarabine, mitoxantrone, idarubicin, dexamethasone, topotecan, pemetrexed, thalidomide, gemcitabine, etoposide, letrozole, leuprorelin, azacitidine and vinorelbine, may be used.

In another aspect, the present invention provides a liposome including the compound.

In another aspect, the present invention provides a pharmaceutical composition for cancer treatment containing the compound or the liposome.

In another aspect, the present invention provides a method for preventing or treating cancer, which includes injecting or administering a composition containing the compound or the liposome to a subject.

In another aspect, the present invention provides a use of a composition containing the compound or the liposome for prevention or treatment of cancer.

### Advantageous Effects of Invention

According to exemplary embodiments of the present invention, it is possible to conduct cancer treatment greatly safely as a lipid-based nano-drug delivery vehicle containing a photosensitizer for photodynamic therapy, a diagnostic and therapeutic radioisotope-conjugated chelator for nuclear medicine diagnosis and therapy or a drug for chemotherapy and an imidazopyridine derivative that can specifically target TSPO is contained to enhance the targeting ability, leading to high binding affinity for biomarkers and to prevent unwanted effects on other organs in the body. According to exemplary embodiments of the present invention, the compound can be effectively used in cancer treatment as a substitute for existing anticancer drugs.

### BRIEF DESCRIPTION OF DRAWINGS

FIG. 1 illustrates the results of a docking simulation of a compound according to an embodiment of the present invention;

FIG. 2A is a graph illustrating the stability of a liposome according to an embodiment of the present invention;

FIG. 2B is images illustrating the stability of a liposome according to an embodiment of the present invention;

FIG. 3 is a graph illustrating the stability of a liposome according to an embodiment of the present invention under various pH conditions;

FIG. 4 is a graph illustrating the pH sensitivity of a liposome according to an embodiment of the present invention;

FIG. 5 is images evaluating the TSPO-specific targeting ability of a liposome according to an embodiment of the present invention;

FIG. 6 is images evaluating the ROS generating ability of a liposome according to an embodiment of the present invention in cancer cells;

FIG. 7 is a graph illustrating the results of in vitro measurement of the photodynamic therapeutic effect of a liposome according to an embodiment of the present invention;

FIG. 8A is a graph illustrating the change in tumor size after photodynamic therapy with a liposome according to an embodiment of the present invention; and

FIG. 8B is a graph illustrating the change in body weight of mice that have undergone photodynamic therapy with a liposome according to an embodiment of the present invention.

### DESCRIPTION OF EMBODIMENTS

The terms used in this specification are selected from the most commonly used terms in current usage taking into account the functions of the present invention, but may vary depending on the intention of engineers in the field, case law, or the emergence of new technologies. Additionally, in certain cases, there are terms arbitrarily selected by the applicant, and in such cases, their meanings will be described in detail in the description of the relevant invention. Therefore, the terms used in this specification should be defined based on the meaning of the terms and the overall content of the present invention, rather than simply the names of the terms.

Unless otherwise defined, all terms used herein, including technical or scientific terms, have the same meaning as commonly understood by those skilled in the art to which the present invention pertains. Generally understood terms should be interpreted as having the same meaning as they have in the context of the relevant technology, and are not to be construed in an ideal or overly formal sense unless expressly defined in the present invention.

The numerical range includes the numerical values defined in the present invention. Any maximum numerical limitation given throughout this specification includes any lower numerical limitation as if that lower numerical limitation were explicitly written out. Any minimum numerical limitation given throughout this specification includes any higher numerical limitation as if that higher numerical limitation were explicitly written out. Any numerical limitation given throughout this specification will include any better numerical range within that broader numerical range as if that the narrower numerical limitation were explicitly written out.

As used herein, the words “including,” “having,” and “containing” are inclusive or open-ended and do not exclude additional unrecited elements or method steps. The term “or any combination thereof” as used herein refers to all permutations and combinations of the items listed before the term. For example, “A, B, C, or any combination thereof” is intended to include at least one of A, B, C, AB, AC, BC or ABC, and, where order is important in a particular context, BA, CA, CB, CBA, BCA, ACB, BAC or CAB. Along with this example, combinations containing repetitions of one or more items or terms, for example, BB, AAA, MB, BBC, AAABCCCC, CBBAAA, CABABB and the like may be included. Those skilled in the art will understand that, unless the context clearly indicates otherwise, there is typically no limit to the number of items or terms in an arbitrary combination.

The term “treatment” as used herein shall be given the broadest meaning unless otherwise specified, and includes the resolution, treatment, management, alleviation, cure, and prevention of conditions and diseases, including conditions and diseases of animals, mammals and humans, and any combination or modification thereof.

The term “photodynamic therapy (PDT)” as used herein refers to a next-generation treatment method in which a photosensitizer is activated by light and then chemically reacts with molecular oxygen to generate reactive oxygen species (ROS) and the reactive oxygen species selectively destroy targeted cells or tissues.

The term “radiodiagnosis and radiotherapy” as used herein refers to the diagnostic and therapeutic methods in the nuclear medicine field in which nuclear medicine diagnosis through positron emission tomography (PET) using positron emission of radioisotopes or single photon emission computed tomography (STE) using the emission of single photons is conducted and cancer treatment using the energy of alpha or beta emitters emitted from radioisotopes is conducted.

The term “chemotherapy” as used herein refers to modern chemotherapy in which functional impairment, nuclear damage and the like of cancer cells are caused using compounds to treat cancer.

The term “biomarker” as used herein refers to a substance that can indicate the presence or risky state of a disease. As an example, in the case of cancer diagnosis, a “biomarker” may refer to a substance that indicates the presence or risk of cancer. A “biomarker” may include a polypeptide and a protein, the amount of which is larger or smaller in a patient suffering from cancer or at risk for developing cancer compared to a normal healthy subject.

The term “ligand” as used herein refers to a substance that selectively binds to the biomarker. Types of ligands that can be used include antibodies, proteins, peptides, and other low molecular weight compounds.

The term “cancer” as used herein refers to abnormal and uncontrolled cell proliferation in the body, and is also called a “malignant tumor.” Cancer mentioned in exemplary embodiments of the present invention include ocular cancer, rectal cancer, colorectal cancer, pituitary cancer, adrenal cancer, prostate cancer, breast cancer, bladder cancer, esophageal cancer, laryngeal cancer, oral cancer, stomach cancer, liver cancer, colon cancer, rectal cancer, pancreatic cancer, liver cancer, gallbladder cancer, cholangiocarcinoma, lung cancer, skin cancer, kidney cancer, vaginal cancer, vulvar cancer, cervical cancer, uterine cancer, ovarian cancer, ovarian cancer, testicular cancer, kidney cancer, brain cancer (for example, glioma), throat cancer, skin melanoma, acute lymphoblastic leukemia, acute myeloid leukemia, Ewing's sarcoma, Kaposi's sarcoma, basal cell carcinoma, squamous cell carcinoma, small cell lung cancer, choriocarcinoma, rhabdomyosarcoma, hemangiosarcoma, hemangioendothelioma, Wilms' tumor, neuroblastoma, lymphoma, myeloma, neurofibromatosis, tuberous sclerosis, Waldenstrom macroglobulinemia, monoclonal gammopathy, benign monoclonal gammopathy, heavy-chain disease, bone and connective tissue sarcomas, brain tumors, thyroid cancer, hemangiomatosis, and lymphangiogenesis, but are not limited thereto.

Hereinafter, exemplary embodiments of the present invention will be described in detail. However, it is obvious that the present invention is not limited to the following exemplary embodiments.

In an aspect, in exemplary embodiments of the present invention, provided is a compound represented by the following Chemical Formula 1, which includes a drug and an imidazopyridine derivative.

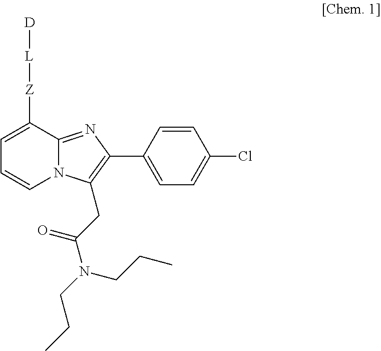

In the formula, D is a drug; L is a linker; and Z is a bonding group (conjugation).

Z may be selected from the group consisting of an ether, an amide, an ester, urea, urethane, thiourea, a sulfide and a disulfide. For example, Z may be

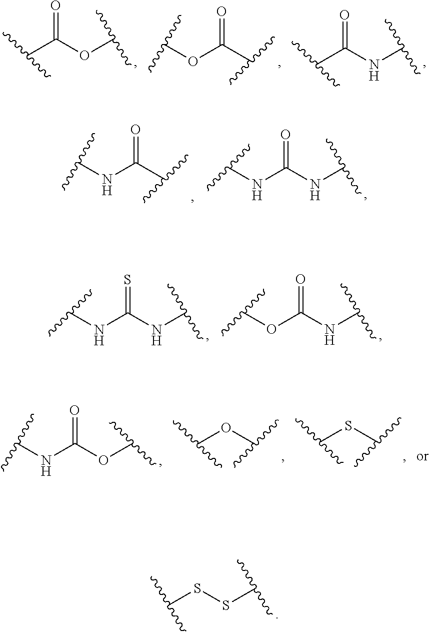

L may be -(PEG)n-, —(CH₂)n- or -phenyl-, and n may be 0 to 20.

In an aspect, the drug may include a photosensitizer for the purpose of performing photodynamic therapy (PDF). For example, the photosensitizer may include one or more selected from the group consisting of porphyrin derivatives, chlorin derivatives, phthalocyanine derivatives, merocyanine derivatives, porphycene derivatives, heptamethine cyanine derivatives, chitosan derivatives, methylene blue derivatives, monoterpene derivatives, xanthene derivatives, toluidine blue derivatives, fluorescein derivatives, and menaquinone derivatives.

Examples of the porphyrin derivatives include 5-aminolaevulinic acid, protoporphyrin IX, verteporfin, photofrin, benzoporphyrin, verteporfin, and phthalocyanine Pc 4, examples of the chlorin derivatives include purlytin, foscan, tetra(m-hydroxyphenyl)chlorin, bacteriochlorin, foscan, mono-aspartyl chlorin e6, and lutetium texaphyrin, and examples of the phthalocyanine derivatives include zinc(III) phthalocyanine, sulfonated zinc(II), and Al(III) phthalocyanine chloride tetrasulfonic acid. Examples of the merocyanine derivatives include MC540 and rhodamine complex, examples of the heptamethine cyanine derivatives include MHI-148, IR780, IR-783, IR-786, IR-808, IR-813, R820, ICG, Pz 247, and MHI-148-783, and examples of the chitosan derivatives include sodium tripolyphosphate and tetrasodium pyrophosphate decahydrate. Examples of the methylene blue derivatives include methylene blue, dimethylene blue, and new methylene blue, examples of the monoterpene derivatives include azulene, examples of the xanthene derivatives include erythrosin, and examples of the toluidine blue derivatives include toluidine blue 0.

In an aspect, the drug may include a metal chelator labeled with a metallic radioisotope for the purpose of performing radiodiagnosis and radiotherapy. For example, the metallic radioisotope may include one or more selected from the group consisting of ⁶⁰ Cu, ⁶¹Cu, ⁶²Cu, ⁶⁴Cu, ⁶⁷Cu, ⁶⁶Ga, ⁶⁷Ga, ⁶⁸Ga, ⁴⁴Sc, ⁴⁷SC, ¹¹¹In, ^{114m}In, ¹¹⁴In, ⁸⁶Y, ⁹⁰Y, ²¹²Bi, ²¹³Bi, ²¹²Pb, ²²⁵Ac, ⁸⁹Zr and ¹⁷⁷Lu, and the metal chelator may include one or more selected from the group consisting of DOTA (1,4,7,10-tetraazacyclododecane-1,4,7,10-tetraacetic acid), DOTAGA, CB-DO2A (4,10-bis(carboxymethyl)-1,4,7,10-tetraazabicyclo[5.5.2]tetradecane), TCMC (1,4,7,10-tetrakis(carbamoylmethyl)-1,4,7,10-tetraazacyclododecane), 3p-C-DEPA, p-NH2-Bn-Oxo-DO3A, TETA (1,4,8,11-tetraazacyclotetradecane-1,4,8,11-tetraacetic acid), CB-TE2A (4,11-bis-(carboxymethyl)-1,4,8,11-tetraazabicyclo[6.6.2]-hexadecane), Diamasar, NOTA (1,4,7-triazacyclononane-1,4,7-triacetic acid), p-SCN-Bn-NOTA (C-NOTA), NETA ({4-[2-(bis-carboxymethylamino)-ethyl]-7-carboxymethyl-[1,4,7]triazonan-1-yl}-acetic acid), TACN-TM (N,N′,N″, tris(2-mercaptoethyl)-1,4,7-triazacyclononane), DTPA (diethylenetriaminepentaacetic acid), CHX-A″-DTPA (2-(p-isothiocyanatobenzyl)-cyclohexyldiethylenetriaminepentaacetic acid), TRAP ((PRP9, TRAP-Pr), 1,4,7-triazacyclononane-1,4,7-tris[methyl(2-carboxyethyl)phosphinic acid]), AAZTA (1,4-bis(hydroxycarbonyl methyl)-6-[bis(hydroxylcarbonyl methyl)]amino-6-methyl perhydro-1,4-diazepine), H2dedpa (1,2-[[6-(carboxy)-pyridin-2-yl]-methylamino]ethane), H4octapa (N,N′-bis(6-carboxy-2-pyridylmethyl)-ethylenediamine-N,N′-diacetic acid), H2azapa (N,N′-[1-benzyl-1,2,3-triazole-4-yl]methyl-N,N′-[6-(carboxy)pyridin-2-yl]-1,2-diaminoethane), H5decapa (N,N″-[[6-(carboxy)pyridin-2-yl]methyl]-diethylenetriamine-N,N′,N″-triacetic acid), HBED (N,N′-bis(2-hydroxybenzyl)-ethylenediamine-N,N′-diacetic acid), SHBED (N,N′-bis(2-hydroxy-5-sulfobenzyl)-ethylenediamine-N,N′-diacetic acid), BPCA, CP256, PCTA (3,6,9,15-tetraazabicyclo[9.3.1]-pentadeca-1(15), 11,13-triene-3,6,9, -triacetic acid), DFO (desferrioxamine B), p-SCN-Bn-DFO, H6phospa (N,N′-(methylenephosphonate)-N,N′-[6-(methoxycarbonyl)pyridin-2-yl]-methyl-1,2-diaminoethane), HEHA (1,4,7,10,13,16-hexaazacyclohexadecane-N,N′,N″,N′″,N″ ″,N′″″-hexaacetic acid) and PEPA (1,4,7,10,13-pentaazacyclopentadecane-N,N′,N″,N′″,N″ ″-pentaacetic acid).

The metal chelator labeled with a metallic radioisotope may be, for example, a DOTA metal chelator labeled with ⁶⁰ Cu, ⁶¹Cu, ⁶²Cu, ⁶⁴Cu, ⁶⁷Cu, ⁶⁷Ga, ⁶⁸Ga, ⁴⁴Sc, ⁴⁷Sc, ¹¹¹In, ¹⁷⁷Lu ⁸⁶ Y, ⁹⁰Y, ²¹³Bi, ²¹²Pb or ²²⁵Ac; a CB-DO2A metal chelator labeled with ⁶⁰Cu, ⁶¹Cu, ⁶²Cu, ⁶⁴Cu, ⁶⁷Cu, ⁶⁷Ga or ⁶⁸Ga; a TCMC metal chelator labeled with ²¹²Pb; a 3p-C-DEPA metal chelator labeled with ²¹²Bi or ²¹³Bi; a TETA metal chelator labeled with ⁶⁰Cu, ⁶¹Cu, ⁶²Cu, ⁶⁴Cu or ⁶⁷Cu; a CB-TE2A metal chelator labeled with ⁶⁰Cu, ⁶¹Cu, ⁶²Cu, ⁶⁴Cu or ⁶⁷Cu; Diamsar labeled with ⁶⁰Cu, ⁶¹Cu, ⁶²Cu, ⁶⁴Cu or ⁶⁷Cu; a NOTA metal chelator labeled with ⁶⁰Cu, ⁶¹Cu, ⁶²Cu, ⁶⁴Cu, ⁶⁷Cu, ⁶⁷Ga or ⁶⁸Ga; a NETA metal chelator labeled with ¹⁷⁷Lu, ⁸⁶Y, ⁹⁰Y, ²¹²Bi or ²¹³Bi; a DTPA metal chelator labeled with ⁴⁴Sc, ⁴⁷Sc, ¹¹¹In, ¹⁷⁷Lu, ⁸⁶Y or ⁹⁰Y; a CHX-A″-DTPA metal chelator labeled with ¹¹¹In, ¹⁷⁷Lu ⁸⁶, Y⁹⁰Y or ²¹³Bi; a TRAP metal chelator labeled with ⁶⁷Ga or ⁶⁸Ga; an AAZTA metal chelator labeled with ⁶⁷Ga or ⁶⁸Ga; a H2dedpa metal chelator labeled with ⁶⁰Cu, ⁶¹Cu, ⁶²Cu, ⁶⁴Cu, ⁶⁷Cu, ⁶⁷Ga or ⁶⁸Ga; an H4octapa metal chelator labeled with ¹¹¹In or ¹¹⁷Lu; an H2azapa metal chelator labeled with ⁶⁰Cu, ⁶¹Cu, ⁶²Cu, ⁶⁴Cu or ⁶⁷Cu; an HBED metal chelator labeled with ⁶⁷Ga or ⁶⁸Ga; an SHBED metal chelator labeled with ⁶⁷Ga, ⁶⁸Ga, or ¹¹¹In; a BPCA metal chelator labeled with 1¹¹¹In; a CP256 metal chelator labeled with ⁶⁷Ga or ⁶⁸Ga; a PCTA metal chelator labeled with ⁶⁰Cu, ⁶¹Cu, ⁶²Cu, ⁶⁴Cu, ⁶⁷Cu, ⁶⁷Ga or ⁶⁸Ga; a DFO metal chelator labeled with ⁶⁷Ga, ⁶⁸Ga or ⁸⁹Zr; or an H6phospa metal chelator labeled with ⁸⁹Zr. In this way, composites selected from the group of metallic radioisotope-labeled chelators that can be subjected to cancer diagnosis or radiotherapy through in vivo nuclear medicine examination such as positron emission tomography (PET) and single photon emission computed tomography (SPECT) may be used.

In an aspect, the drug may include a chemotherapeutic drug for the purpose of performing chemotherapy. As the chemotherapeutic drug, for example, one or more selected from the group consisting of representative anticancer drugs known as chemotherapeutic drugs, such as doxorubicin, hydroxyurea, vincristine, docetaxel, cyclophosphamide, carboplatin, methotrexate, paclitaxel, cisplatin, 5-fluorouracil, leucovorin, prednisolone, melphalan, chlorambucil, carmustine, daunorubicin, bleomycin, cytarabine, busulfan, capecitabine, 5-FU, mitomycin C, tamoxifen, bicalutamide, gonadotropin, irinotecan, belotecan, ifosfamide, temozolomide, fludarabine, mitoxantrone, idarubicin, dexamethasone, topotecan, pemetrexed, thalidomide, gemcitabine, etoposide, letrozole, leuprorelin, azacitidine and vinorelbine, may be used.

In an embodiment, the compound may be represented by the following Chemical Formula 2. The compound represented by Chemical Formula 2 is an effective compound targeting mitochondria in cancer cells, in which a representative photosensitizer IR780 is conjugated to an imidazopyridine derivative.

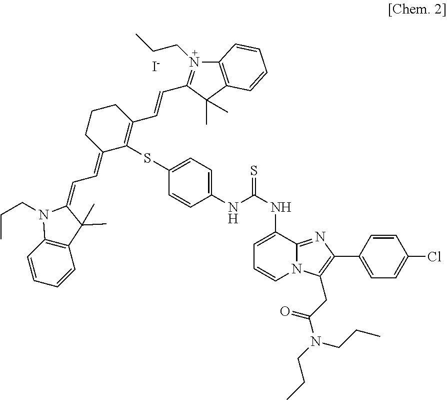

The compound represented by Chemical Formula 2 may be prepared by the following method: as shown in the following Reaction Scheme 1, a step of reacting chlorobenzoyl propionic acid with 1,1′-carbonyldiimidazole, triethylamine, and dipropylamine in a dimethylformamide solvent to prepare 4-(4-chlorophenyl)-4-oxo-N,N-dipropylbutanamide (step 1); a step of reacting 4-(4-chlorophenyl)-4-oxo-N,N-dipropylbutanamide with bromine in a chloroform solvent to prepare 3-bromo-4-(4-chlorophenyl)-4-oxo-N,N-dipropylbutanamide (step 2); a step of reacting 3-bromo-4-(4-chlorophenyl)-4-oxo-N,N-dipropylbutanamide with 2-amino-3-nitro pyridine in a dimethylformamide solvent to prepare 2-(2-(4-chlorophenyl)-8-nitroimidazo[1,2-a]pyridin-3-yl)-N,N-dipropylacetamide (step 3); a step of reacting 2-(2-(4-chlorophenyl)-8-nitroimidazo[1,2-a]pyridin-3-yl)-N,N-dipropylacetamide with zinc powder and ammonium chloride in a 90% water-methanol mixed solution to prepare 2-(8-amino-2-(4-chlorophenyl)-imidazo[1,2-a]pyridin-3-yl)-N,N-dipropylacetamide (step 4); a step of reacting 2-(8-amino-2-(4-chlorophenyl)-imidazo[1,2-a]pyridin-3-yl)-N,N-dipropylacetamide with di(2-pyridyl) thiocarbonate in a dichloromethane solvent to prepare 2-(2-(4-chlorophenyl)-8-isothiocyanatoimidazo[1,2-a]pyridin-3-yl)-N,N-dipropylacetamide (step 5); and a step of reacting 2-(2-(4-chlorophenyl)-8-isothiocyanatoimidazo[1,2-a]pyridin-3-yl)-N,N-dipropylacetamide with IR780-NH₂ in a dimethylformamide solvent to prepare an IR780-bound compound targeting mitochondria in cancer cells (step 6).

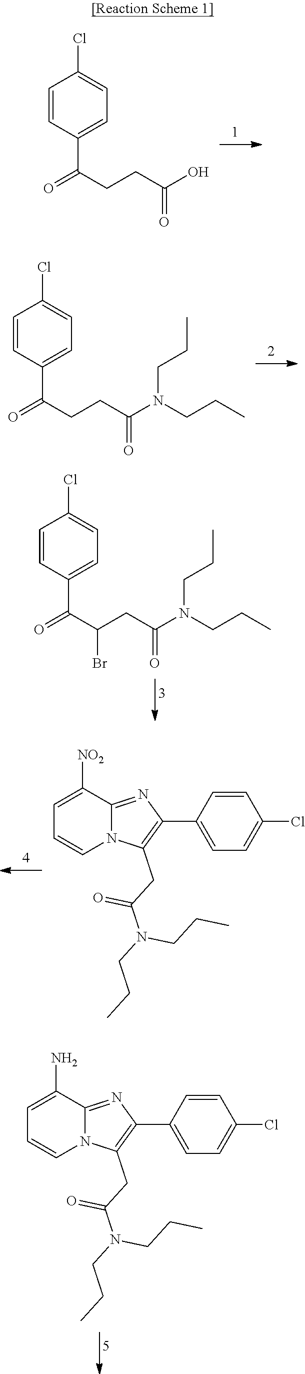 [SMILES: O=C(O)CCC(=O)c1ccc(Cl)cc1 ; CCCN(CCC)C(=O)CCC(=O)c1ccc(Cl)cc1 ; CCCN(CCC)C(=O)CC(Br)C(=O)c1ccc(Cl)cc1 ; CCCN(CCC)C(=O)Cc1c(-c2ccc(Cl)cc2)nc2c([N+](=O)[O-])cccn12 ; CCCN(CCC)C(=O)Cc1c(-c2ccc(Cl)cc2)nc2c(N)cccn12]

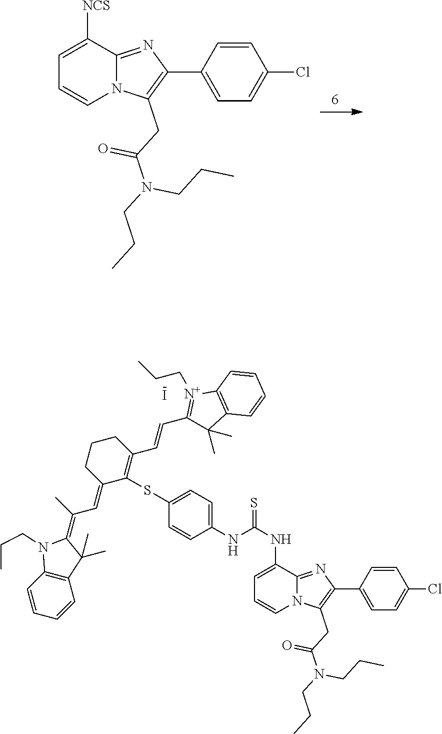

In Reaction Scheme 1 according to the present invention, the intermediate products obtained at each step may be separated/purified through a filtration method, purification method and the like known in the field of organic synthesis.

In an embodiment, the photosensitizer in Chemical Formula 2 may be IR780 represented by the following Chemical Formula 3. IR780 acts as a compound that generates reactive oxygen species when exposed to light with a specific wavelength (780 to 800 nm), and thus, in cancer treatment, treatment in which tumor at a targeted site is killed by reactive oxygen species generated by irradiating the site with light is possible.

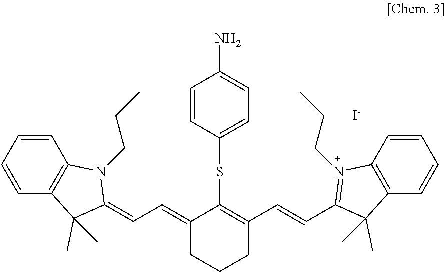

Korean Patent No. 10-2031652 discloses a compound (represented by the following Chemical Formula 4) in which IR-780 is introduced into a 2-aryl-6,8-dichloroimidazopyridine derivative, but this compound does not bind to TSPO because of steric hindrance as a result of molecular docking simulation and is thus not suitable for cancer treatment targeting TSPO.

However, the compound represented by Chemical Formula 1 according to an embodiment of the present invention has excellent binding power for TSPO at the nanomolar level (Kᵢ=201 nM for TSPO) without steric hindrance as a photosensitizer is introduced at a specific position.

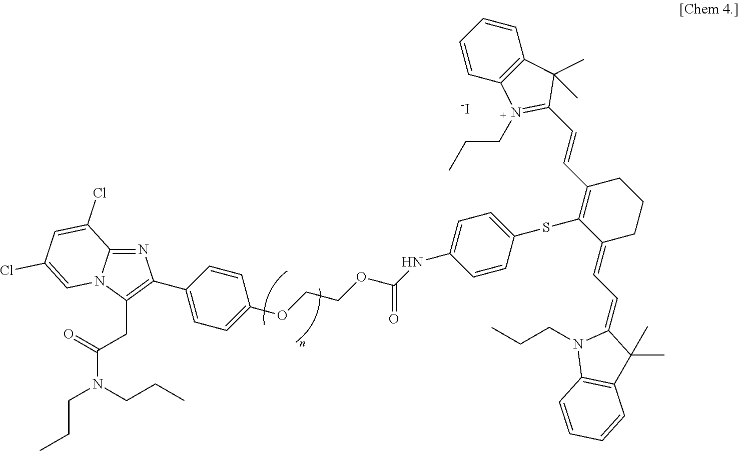

In an embodiment, the compound may be used in photodynamic therapy.

In another aspect, the present invention provides a liposome containing the above-described compound represented by Chemical Formula 1, which contains a drug and an imidazopyridine derivative.

In an embodiment, the liposome may be a pH-sensitive liposome. The pH-sensitive liposome refers to a liposome that collapses at a specific pH, which is the acidic microenvironment of a tumor, releasing the substance inside the liposome. For example, the pH-sensitive liposome may collapse in an environment having a pH of 6.0 to 6.8, releasing the substance inside the liposome.

In an embodiment, the liposome may further contain a drug. The drug may include, for example, one or more selected from the group consisting of docetaxel, cisplatin, camptothecin, paclitaxel, tamoxifen, anasterozole, gleevec, 5-fluorouracil (5-FU), floxuridine, leuprolide, flutamide, zoledronate, doxorubicin, vincristine, gemcitabine, streptozocin, carboplatin, topotecan, belotecan, irinotecan, vinorelbine, hydroxyurea, valrubicin, retinoic acid-based drugs, methotrexate, meclorethamine, chlorambucil, busulfan, doxifluridine, vinblastine, mitomycin, prednisone, testosterone, mitoxantron, aspirin, salicylates, ibuprofen, naproxen, fenoprofen, indomethacin, phenyltazone, cyclophosphamide, mechlorethamine, dexamethasone, prednisolone, celecoxib, valdecoxib, nimesulide, cortisone and corticosteroid.

In another aspect, the present invention provides a pharmaceutical composition for cancer treatment containing the compound or liposome described above.

In another aspect, the present invention provides a method for preventing or treating cancer, which includes injecting or administering a composition containing the compound or liposome described above to a subject.

In another aspect, the present invention provides a use of a composition containing the compound or liposome described above for prevention or treatment of cancer.

In an embodiment, the cancer treatment may be photodynamic therapy.

In an embodiment, the cancer treatment may be radiotherapy.

In an embodiment, the cancer treatment may be chemotherapy.

In an embodiment, the cancer may be a cancer associated with overexpression of translocator protein (TSPO). For example, the cancer may include one or more selected from the group consisting of pancreatic cancer, lung cancer, thyroid cancer, ovarian cancer, uterine cancer, colorectal cancer, stomach cancer, bladder cancer, liver cancer, prostate cancer, breast cancer, kidney cancer and brain cancer.

Hereinafter, the composition and effects of the present invention will be described more specifically with reference to Examples. However, Examples below are provided for illustrative purposes only to help understand the present invention and the scope and range of the present invention are not limited thereby.

### EXAMPLES

### [Example 1] Preparation of IR780-Bound Compound Targeting Mitochondria in Cancer Cells

### 1. Materials

6-maleimidohexanehydrazide trifluoroacetate (EMCH) was purchased from Tokyo Chemical Industry (Tokyo, Japan). IR780, methoxy-PEG (mPEG2000, Mn=2000), cholesterol, thiazolyl blue tetrazolium (MTT), and PK11195 were purchased from Sigma Aldrich (St. Louis, USA). 1,2-dipalmitoyl-sn-glycero-3-phosphothioethanol sodium salt (DPPE), 1,2-distearoyl-sn-glycero-3-phosphocholine (DSPC), and 1,2-distearoyl-sn-glycero-3-phosphoethanolamine-N-[methoxy(polyethylene glycol)-2000](ammonium salt) (18:0 PEG2000 PE) were purchased from Avanti Polar Lipids (Alabaster, MA, USA). Flash column chromatography was performed using silica gel (Merck, 230-400 mesh, ASTM). All reactions were monitored by thin-layer chromatography (Merck, silica gel 60F254). FBS (fetal bovine serum), DMEM (Dulbecco's Modified Eagle Medium) and size exclusion PD-10 column were purchased from Cytiva (Marlborough, MA, USA). 4% PFA (paraformaldehyde) was purchased from BIOSESANG (Seongnam, South Korea). 1,1′-dioctadecyl-3,3,3′,3′-tetramethylindodicarbocyanine, 4-chlorobenzenesulfonate salt (DiD), and MitoTrackerTM Deep Red FM (Mitotracker) were purchased from Invitrogen (Carlsbad, CA, USA). Hoechst 33342 Solution was purchased from Thermo Fisher Scientific (Waltham, MA, USA). ViaFluor® 488 Live Cell Microtubule Staining Kit was purchased from Biotium (Fremont, CA, USA). Female BALB/c nude mice (6 to 8 weeks old) were purchased from ORIENT BIO (Seongnam, Korea).

Electrospray mass spectrometry (ESI-MS) was performed using an Agilent 1100 LC-MSD trap system instrument. The melting point (m.p.) was measured using Buchi apparatus. ¹H and ¹³C NMR spectra were measured under ambient condition using Varian 400-MR spectrometer (Agilent). Chemical shifts were measured in ppm (unit: 6). All hydrodynamic sizes of liposomes were measured using a dynamic light scattering instrument (DLS, ZETASIZER Nano ZS, Malvern Instrument Ltd., Worcestershire, UK). Fluorescence and absorbance signals were measured using a microplate reader (SYNERGY H1, BioTek, Vermont, USA). TSPO targeting and reactive oxygen species (ROS) generation were observed under a confocal microscope (Nikon AIR, Nikon Co., Tokyo, Japan). An 808-nm NIR laser (FC-W-808-10W, CNI, Changchun, China) was used for in vitro and in vivo PDT. In vivo mouse fluorescence images were obtained using an imaging system (IVIS, IVIS Lumina X5 Imaging System, Perkin-Elmer, Waltham, MA, USA).

### 2. Preparation of IR780-Bound Compound Targeting Mitochondria in Cancer Cells

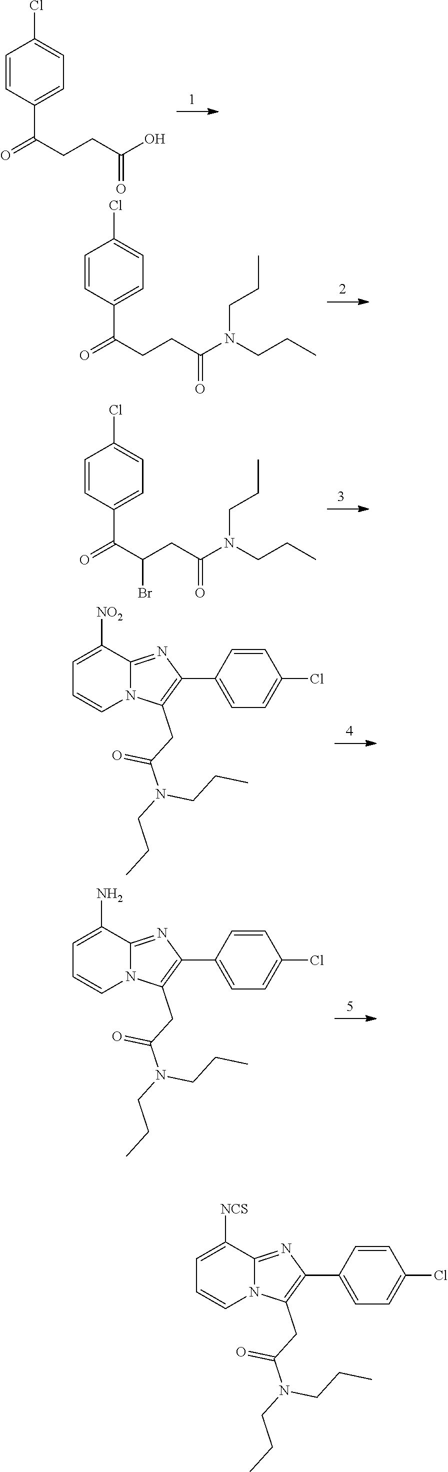

### (Step 1): Preparation of 4-(4-chlorophenyl)-4-oxo-N,N-dipropylbutanamide

In N,N′-dimethylformamide (40 ml), 3-(4-chlorobenzoyl)propionic acid (8.3 g, 38.9 mmol) and 1,1′-carbonyldiimidazole (7.0 g, 42.9 mmol) were dissolved and stirred for 30 minutes, then N,N′-dipropylamine (6.2 mL, 44.7 mmol) and triethylamine (6.8 mL, 48.9 mmol) were added to this mixture, and stirring was performed for 8 hours. The solvent was removed, then 0.5 N hydrogen chloride aqueous solution was added to the remaining mixture, and extraction with dichloromethane (30 mL) was performed two times. The residual water was removed from the extracted organic layer using sodium sulfate, then the solvent was removed, and purification was performed by column chromatography to obtain the desired compound at a yield of 83%.

¹H NMR (400 MHz, CD₃OD) δ 0.88 (t, J=7.6 Hz, 3H), 0.99 (t, J=7.6 Hz, 3H), 1.55 (sx, J=7.6 Hz, 2H), 1.72 (sx, J=7.6 Hz, 2H), 2.79 (t, J=7.4 Hz, 2H), 3.24-3.42 (m, 4H), 7.51 (d, J=8.4 Hz, 2H), 8.01 (d, J=8.8 Hz, 2H); ¹³C NMR (100 MHz, CDCl₃) δ 11.5, 21.1, 22.3, 27.3, 34.0, 48.0, 49.8, 129.0, 129.7, 135.4, 139.6, 171.3, 198.4; MS (ESI) m/z 296.1 (M+H)⁺

### (Step 2): Preparation of 3-bromo-4-(4-chlorophenyl)-4-oxo-N,N-dipropylbutanamide

After 4-(4-chlorophenyl)-4-oxo-N,N-dipropylbutanamide (3.7 g, 12.5 mmol) prepared in step 1 was dissolved in chloroform (30 mL), bromine (0.8 mL, 15 mmol) was slowly added. The reaction mixture was stirred at 50° C. for 90 minutes for reaction. After the reaction, the temperature of the mixture was lowered to room temperature, the residual bromine was removed using 1 μM sodium thiosulfate (30 mL) and water (30 mL), and then the residual water was removed using sodium sulfate. The mixture from which the residual water had been removed was concentrated under negative pressure and then purified by column chromatography to obtain the desired compound at a yield of 87%.

¹H NMR (400 MHz, CD₃OD) δ 0.83 (t, J=7.6 Hz, 3H), 0.99 (t, J=7.6 Hz, 3H), 1.44-1.78 (m, 4H), 3.04 (dd, J=4.8, 16.0 Hz, 1H), 3.10-3.34 (m, 4H), 3.57 (dd, J=10.0, 16.0 Hz, 1H), 5.60 (dd, J=3.6, 10.0 Hz, 1H), 7.45 (d, J=8.8 Hz, 2H), 7.99 (d, J=8.8 Hz, 2H); ¹³C NMR (100 MHz, CDCl₃) δ 11.4, 21.0, 22.2, 38.5, 40.7, 47.6, 49.7, 129.2, 130.5, 132.8, 140.2, 169.3, 192.2; MS (ESI) m/z 374.1 (M+H)⁺

### (Step 3): Preparation of 2-(2-(4-chlorophenyl)-8-nitroimidazo[1,2-a]pyridin-3-yl)-N,N-dipropylacetamide

After 3-bromo-4-(4-chlorophenyl)-4-oxo-N,N-dipropylbutanamide (4.0 g, 10.7 mmol) prepared in step 2 and 2-amino-3-nitro pyridine (1.9 g, 13.9 mmol) were dissolved in N,N′-dimethylformamide (30 mL), stirring was performed at 150° C. for 16 hours for reaction. After the reaction, the mixture was concentrated under negative pressure and purified by column chromatography to obtain the desired compound at a yield of 11%.

¹H NMR (400 MHz, (CD₃)₂SO) δ 0.81 (t, J=7.6 Hz, 3H), 0.89 (t, J=7.6 Hz, 3H), 1.50 (sx, J=7.6 Hz, 2H), 1.62 (sx, J=8.0 Hz, 2H), 3.25 (t, J=7.6 Hz, 2H), 3.34 (t, J=8.0 Hz, 2H), 7.17 (t, J=7.6 Hz, 1H), 7.57 (d, J=8.8 Hz, 2H), 7.68 (d, J=8.4 Hz, 2H), 8.32 (d, J=7.6 Hz, 1H), 8.69 (d, J=6.8 Hz, 1H); ¹³C NMR (100 MHz, (CD₃)₂SO) δ 11.0, 11.2, 20.5, 21.7, 28.8, 47.1, 49.0, 110.0, 118.9, 123.8, 128.8, 129.7, 131.6, 132.4, 133.0, 136.3, 136.4, 143.1, 167.1; MS (ESI) m/z 415.2 (M+H)⁺

### (Step 4): Preparation of 2-(8-amino-2-(4-chlorophenyl)-imidazo[1,2-a]pyridin-3-yl)-N,N-dipropylacetamide

A mixture prepared by adding 2-(2-(4-chlorophenyl)-8-nitroimidazo[1,2-a]pyridin-3-yl)-N,N-dipropylacetamide (110 mg, 0.27 mmol) prepared in step 3, ammonium chloride (32 mg, 0.60 mmol), and zinc powder (706 mg, 10.8 mmol) to 10% water-methanol (10 mL) was stirred at 45° C. for 5 hours for reaction. After the reaction, the temperature of the mixture was lowered to room temperature, dichloromethane (10 mL) was added, and then the remaining zinc powder was removed through filtration. The mixture from which the zinc powder had been removed was concentrated in a vacuum and purified by column chromatography to obtain the desired compound at a yield of 79%.

¹H NMR (400 MHz, (CD₃)₂SO) δ 0.80 (t, J=7.6 Hz, 3H), 0.83 (t, J=7.6 Hz, 3H), 1.48 (sx, J=7.6 Hz, 2H), 1.56 (sx, J=7.6 Hz, 2H), 3.23 (t, J=7.6 Hz, 2H), 3.32 (t, J=7.6 Hz, 2H), 4.13 (s, 2H), 5.63 (s, 2H), 6.29 (d, J=7.6 Hz, 1H), 6.67 (t, J=7.2 Hz, 1H), 7.44 (d, J=7.2 Hz, 1H), 7.51 (d, J=8.8 Hz, 2H), 7.67 (d, J=8.8 Hz, 2H); ¹³C NMR (100 MHz, (CD₃)₂SO) δ 11.0, 11.2, 20.5, 21.7, 29.3, 47.0, 48.9, 100.2, 112.5, 113.4, 116.7, 128.4, 129.3, 131.8, 133.9, 137.0, 138.2, 139.6, 167.5; MS (ESI) m/z 385.2 (M+H)⁺

### (Step 5): Preparation of 2-(2-(4-chlorophenyl)-8-isothiocyanatoimidazo[1,2-a]pyridin-3-yl)-N,N-dipropylacetamide

Dichloromethane (5 mL) in which di(2-pyridyl) thiocarbonate (46.3 mg, 0.20 mmol) was dissolved was slowly added to a mixture prepared by dissolving 2-(8-amino-2-(4-chlorophenyl)-imidazo[1,2-a]pyridin-3-yl)-N,N-dipropylacetamide (60 mg, 0.16 mmol) prepared in step 4 in dichloromethane (5 mL). The prepared mixture was stirred at room temperature for 12 hours for reaction, and after the reaction, the mixture was concentrated by removing the solvent under negative pressure. The concentrated mixture was purified by column chromatography to obtain the desired compound at a yield of 91%.

¹H NMR (400 MHz, (CD₃)₂SO) δ 0.81 (t, J=7.2 Hz, 3H), 0.87 (t, J=7.2 Hz, 3H), 1.49 (sx, J=7.2 Hz, 2H), 1.61 (sx, J=7.6 Hz, 2H), 3.24 (t, J=7.6 Hz, 2H), 3.35 (t, J=7.6 Hz, 2H), 4.27 (s, 2H), 6.96 (t, J=7.6 Hz, 1H), 7.33 (d, J=7.6 Hz, 1H), 7.56 (t, J=8.4 Hz, 2H), 7.68 (d, J=8.4 Hz, 2H), 8.25 (d, J=2.4 Hz, 1H); ¹³C NMR (100 MHz, (CD₃)₂SO) δ 11.0, 11.2, 20.5, 21.7, 29.0, 47.0, 49.0, 111.4, 119.0, 119.3, 119.4, 124.8, 128.7, 129.4, 132.6, 132.8, 140.4, 141.3, 141.4, 167.1; MS (ESI) m/z 427.1 (M+H)⁺

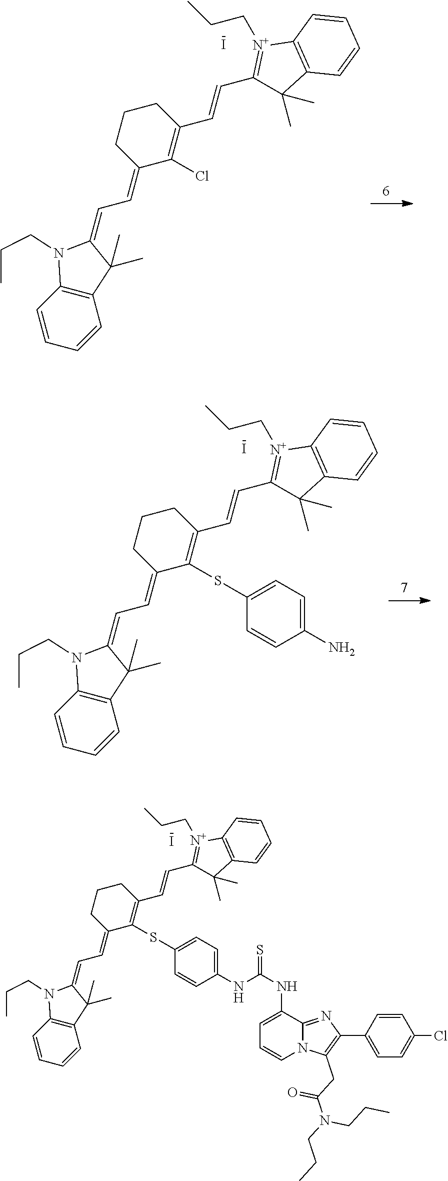

### (Step 6): Preparation of IR780-NH₂

To a mixture prepared by dissolving IR780 (500 mg, 0.75 mmol) in N,N′-dimethylformamide (20 mL), 4-aminothiophenol (0.19 g, 1.49 mmol) was added, and stirring was performed at room temperature for 24 hours for reaction. After the reaction, the mixture was concentrated by removing the solvent under negative pressure and purified by column chromatography to obtain the desired compound at a yield of 80%.

¹H NMR (400 MHz, CD₃OD) δ 1.04 (t, J=7.2 Hz, 6H), 1.57 (s, 12H), 1.82-1.92 (m, 4H), 1.96-2.04 (m, 2H), 2.74 (t, J=7.0 Hz, 4H), 4.11 (t, J=7.6 Hz, 4H), 6.28 (d, J=14.0 Hz, 2H), 6.64 (d, J=8.8 Hz, 2H), 7.02 (d, J=8.8 Hz, 2H), 7.20-7.32 (m, 4H), 7.38-7.49 (m, 4H), 8.86 (d, J=10.0 Hz, 2H); ¹³C NMR (100 MHz, (CD₃)₂SO) δ 11.1, 20.5, 25.8, 27.3, 45.0, 48.8, 101.8, 111.5, 122.4, 125.0, 127.3, 128.5, 133.1, 141.0, 142.2, 144.9, 172.1; MS (ESI) m/z 628.4 (M−I)⁺

### (Step 7): Preparation of IR780-Bound Compound Targeting Mitochondria in Cancer Cells

In N,N′-dimethylformamide (1.0 mL), 2-(2-(4-chlorophenyl)-8-isothiocyanatoimidazo[1,2-a]pyridin-3-yl)-N,N-dipropylacetamide (35 mg, 80.0 mol) obtained in step 5 and IR780-NH₂ (30 mg, 40.0 mol) obtained in step 6 were dissolved and stirred at 80° C. for 90 minutes for reaction. The mixture obtained after the reaction was slowly added into a tube containing diethyl ether (10 mL) and then stored frozen at −20° C. for 5 hours. Afterwards, the solid generated from the mixture was separated using a centrifuge (3400 rpm, 8 minutes), thereby obtaining the desired compound at a yield of 74%.

¹H NMR (400 MHz, CDCl₃) δ 0.76 (t, J=7.6 Hz, 3H), 0.83 (t, J=7.6 Hz, 3H), 1.05 (t, J=7.6 Hz, 6H), 1.47 (s, 12H), 1.46-1.58 (m, 4H), 1.82-1.96 (m, 4H), 2.00-2.18 (m, 2H), 2.72-2.78 (m, 4H), 3.16 (t, J=8.0 Hz, 2H), 3.28 (t, J=7.6 Hz, 2H), 4.06 (t, J=7.6 Hz, 4H), 4.09 (s, 2H), 6.18 (d, J=14.0 Hz, 2H), 6.79 (t, J=7.2 Hz, 1H), 7.09 (d, J=8.0 Hz, 2H), 7.13-7.39 (m, 11H), 7.58-7.64 (m, 4H), 7.93 (d, J=6.8 Hz, 1H), 8.69 (d, J=14.4 Hz, 2H); ³C NMR (100 MHz, (CD₃)₂SO) δ 11.2, 11.5, 11.8, 15.4, 21.0, 21.1, 22.3, 26.8, 28.1, 28.2, 30.5, 46.3, 48.2, 49.5, 50.0, 53.6, 66.0, 101.5, 110.8, 113.0, 116.7, 120.6, 122.5, 125.3, 125.4, 126.6, 128.0, 128.7, 129.0, 130.0, 133.6, 133.9, 134.2, 137.2, 141.3, 142.3, 146.4, 152.2, 167.4, 172.7, 179.1; MS (ESI) m/z 1054.5 (M−I)⁺; HRMS (FAB) m/z (M−I)⁺ calcd for C₆₄H₇₃ClN₇OS₂⁺ 1054.5002, found 1054.5008

### [Example 2] Preparation of Liposome Incorporated with 1R780-Bound Compound Targeting Mitochondria in Cancer Cells

### 1. Preparation of pH-Degradable Lipid

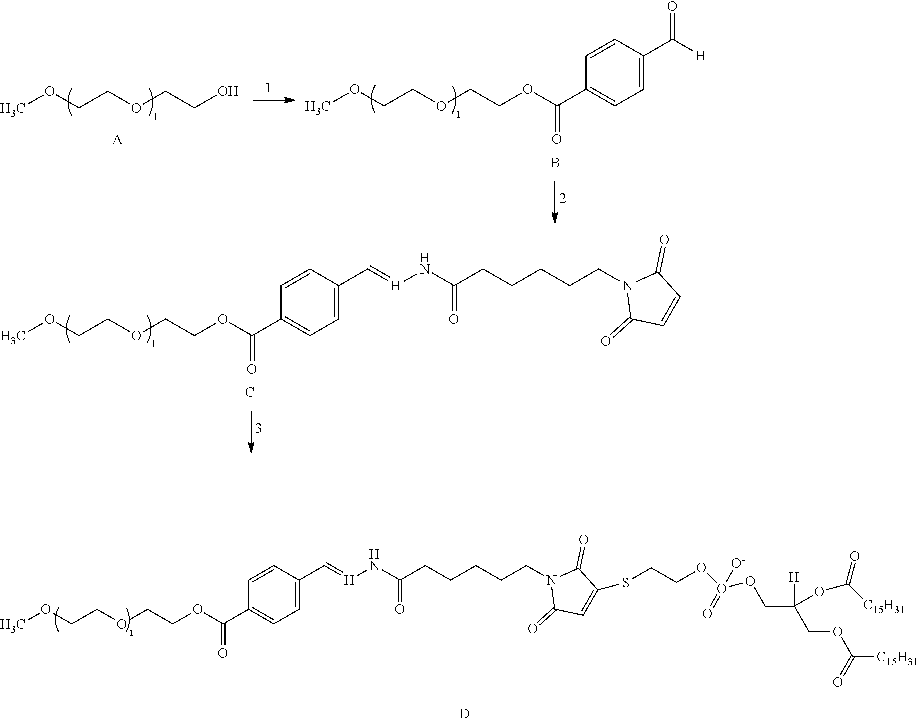

### (Step 1): Preparation of Compound B

To dichloromethane (50 mL) in which mPEG2000 (500 mg, 0.25 mmol) was dissolved, 4-carboxaldehyde (370 mg, 2.5 mmol), 4-(dimethylamino)pyridine (76 mg, 0.63 mmol), and N,N′-dicyclohexylcarbodiimide (500 mg, 2.5 mmol) were added, and stirring was performed at room temperature for 24 hours. After the reaction, the mixture was filtered to remove the solid, the solvent was removed under negative pressure, and then purification was performed by column chromatography to obtain the desired compound at a yield of 84%.

### (Step 2): Preparation of Compound C

mPEG2000-Aldehyde (200 mg, 93 mol) obtained in step 1 and N-F-maleimidocaproic acid hydrazide (EMCH, 47 mg, 140 mol) were dissolved in chloroform (10 mL), and then stirred at room temperature for 18 hours for reaction. After the reaction, the mixture was concentrated under negative pressure and purified by column chromatography to obtain the desired compound at a yield of 81%.

### (Step 3): Preparation of Lipid Compound D

Triethylamine (12 L, 84 mol) was added to a mixture prepared by dissolving mPEG2000-EMCH (100 mg, 42 mol) obtained in step 2 and 1,2-dipalmitoyl-sn-glycero-3-phosphothioethanol (46 mg, 63 mol) in methanol (10 mL), and stirring was performed at room temperature for 18 hours for reaction. After the reaction, the mixture was concentrated under negative pressure and obtained the desired compound at a yield of 85% by column chromatography.

### 2. Preparation of pH-Sensitive Liposome (Drug-pSL) Incorporated with IR780-Bound Compound Targeting Mitochondria in Cancer Cells

After DSPC, cholesterol, and lipid compound D (molar ratio=3:1:1) were added to a methanol-chloroform mixed solution, the IR780-bound compound targeting mitochondria in cancer cells (Drug, 0.5 mol) was added to the mixed solution. Most of the solvent was removed by nitrogen gas purging to form a thin lipid membrane. The residual solvent was removed in a vacuum chamber for 24 hours. The lipid membrane was hydrated with purified water and sonicated using an ip-sonifier to obtain a liposome (Drug-pSL) containing the IR780-bound compound targeting mitochondria in cancer cells. Drug-pSL was purified by size exclusion chromatography.

### [Example 3] Preparation of Liposome (Drug-NL) Incorporated with IR780-Bound Compound Targeting Mitochondria in Cancer Cells

A liposome (Drug-NL) containing the IR780-bound compound targeting mitochondria in cancer cells was prepared by the same method as in Example 2 except that 18:0 PEG2000 PE was added instead of the lipid compound D.
[Comparative Example 1] Preparation of pH-Sensitive Liposome (IR780-pSNL) Containing IR780

A pH-sensitive liposome (IR780-pSNL) containing IR780 was prepared by the same method as in Example 2 except that IR780 was used instead of the IR780-bound compound targeting mitochondria in cancer cells.
[Comparative Example 2] Preparation of Liposome (1R780-NL) Containing IR780

A liposome (IR780-NL) containing IR780 was prepared by the same method as in Example 3 except that IR780 was used instead of the IR780-bound compound targeting mitochondria in cancer cells.

### [Test Example 1] Compound Design by Docking Simulation

In order to perform a docking simulation, a homology model of translocator protein was constructed using Prime provided by the Schrodinger suite as software. LigPrep from the Schrodinger suite was used to calculate all ionization states and tautomers of the ligands at pH 7.0±2.0.

The constructed homology model was preprocessed using the protein preparation module in the Schrodinger suite, then BS-F or BS-Ethyl-F was docked into the binding site of the constructed translocator protein homology model, and full flexibilities of the ligand was allowed during the simulation process. The additional precision (XP) protocol and the basic Force Field OPLS_2005 were applied, the number of docking poses was set from 5 k to 50 k, and the energy was kept to be minimized from 0.4 k to 8 k to explore the configurational space of the ligand during the docking simulation. Finally, the binding free energy for each was obtained through the predicted interaction between the translocator protein and the ligand.

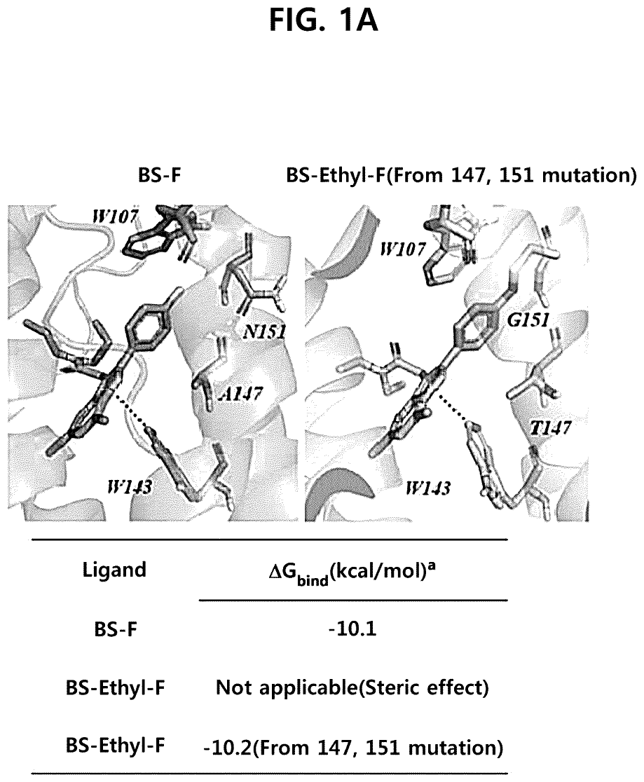

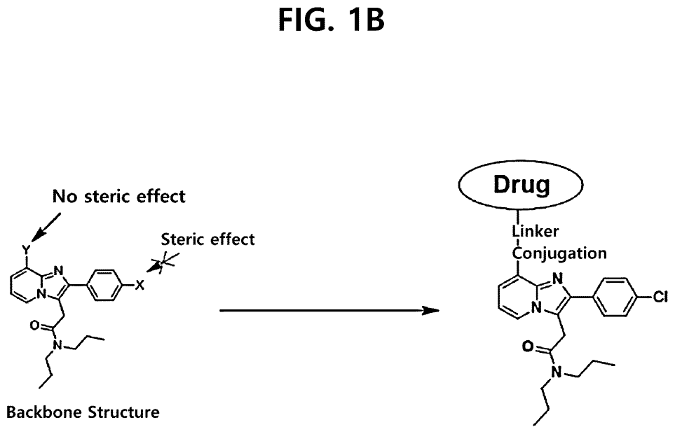

In order to find the conjugation position of IR780, a photosensitizer according to Example of the present invention, and an imidazopyridine derivative, a molecular docking simulation was performed. Specifically, as can be seen from the results in FIG. 1A, it has been found that unlike BS-F, in the case of BS-Ethyl-F, where an ethyl group is introduced into the right benzene ring, the docking simulation does not proceed because of steric hindrance to the amino acid N151 and the docking simulation proceeds only when the amino acid N151 is artificially mutated into G151 for calculation. Accordingly, the compound of the present invention, a compound targeting mitochondria in cancer cells, was designed and synthesized so that IR780 was introduced by selecting a position (position Y in FIG. 1B) where steric hindrance is not expected, and then its protein binding capacity was measured. As a result, it was found that the compound had excellent binding power for TSPO at the nanomolar level (Kᵢ=201 nM for TSPO).

### [Test Example 2] Test of Drug-pSL Stability Under Physiological Condition

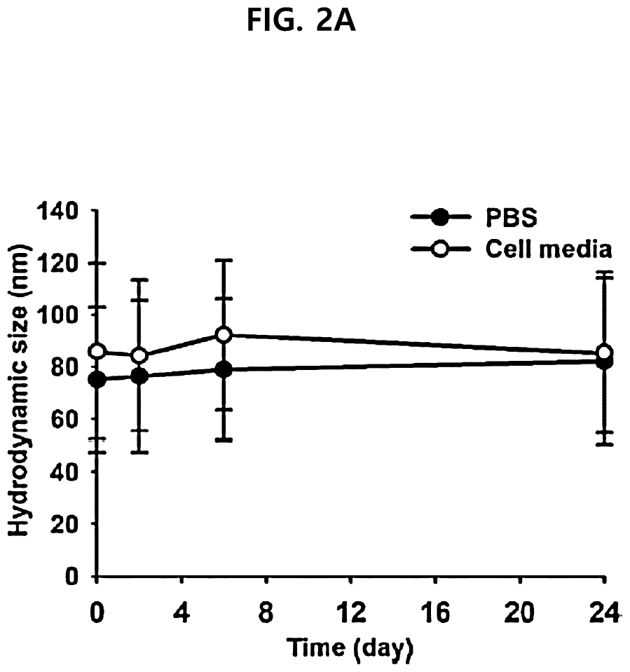

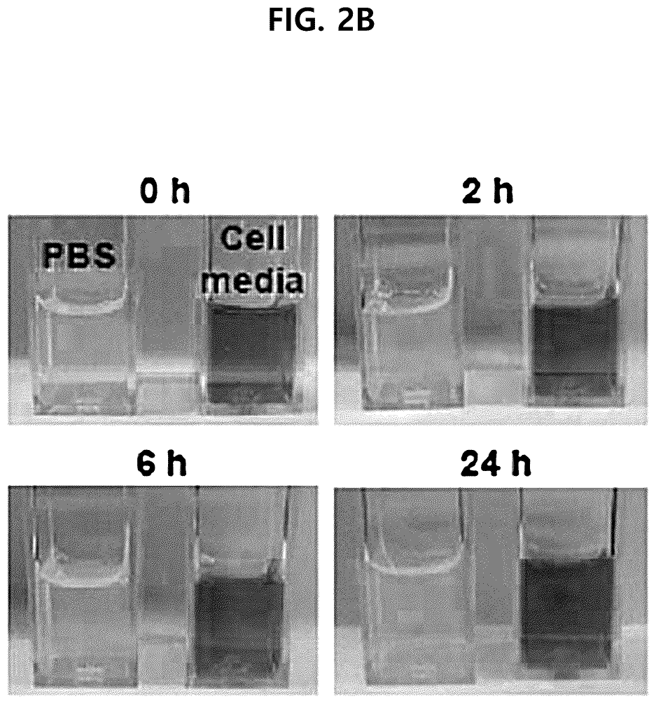

The stability test of Drug-pSL was performed at 37° C. for 24 hours in PBS and a DMEM cell medium containing 10% FBS. The degree of stability under each condition was evaluated at different time points (0, 2, 6, 24 hours) through DLS measurements in terms of hydrodynamic size and is illustrated in FIG. 2A, and the photographic images are illustrated in FIG. 2B.

As can be seen in FIGS. 2A and 2B, Drug-pSL maintained a uniform size in both PBS and DMEM cell medium and did not form visible pellets for 24 hours, indicating high stability.
[Test Example 3] Test of Drug-pSL Stability Under Various pH Conditions

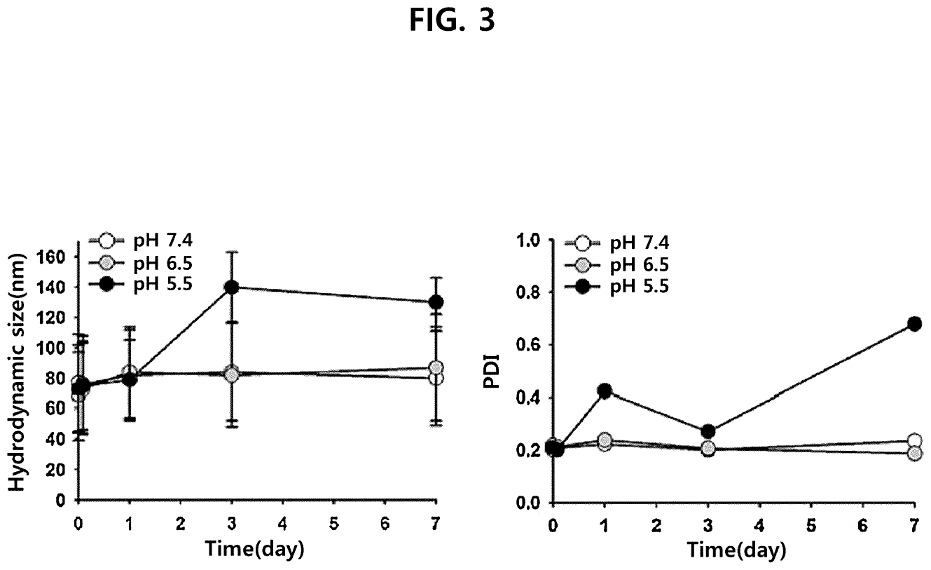

The Drug-pSL stability was performed for 7 days under three different pH conditions (pH 7.4, pH 6.5, and pH 5.5 in 10 mM PBS). The hydrodynamic size and PDI values were measured at various time points (days 0, 1, 3, and 7) and are illustrated in FIG. 3.

As illustrated in FIG. 3, Drug-pSL maintained a uniform size at pH 6.5 and 7.4, while the size increased to 140.5±23.03 nm on day 3 at pH 5.5.

### [Test Example 4] Comparison of pH Sensitivity Between Drug-NL and Drug-pSL Under Various pH Conditions

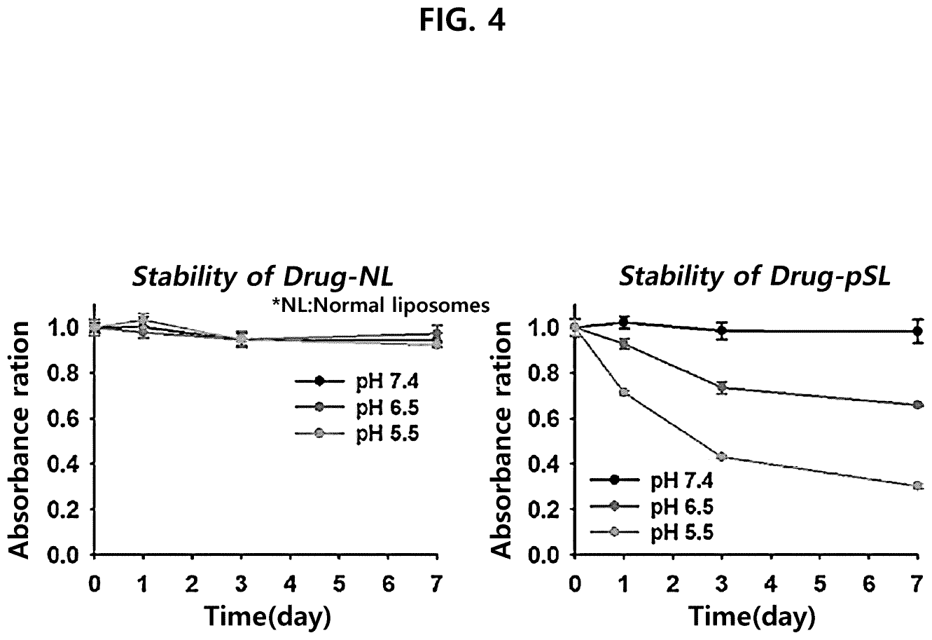

The pH sensitivity was analyzed by the change in absorbance of the IR780-bound compound targeting mitochondria in cancer cells due to liposome degradation for up to 7 days. The pH sensitivity of Drug-NL and Drug-pSL was measured under pH 7.4, pH 6.5, and pH 5.5 conditions in 10 mM PBS for 7 days (days 0, 1, 3, and 7), and the results are illustrated in FIG. 4.

As illustrated in FIG. 4, Drug-NL was stable under all pH conditions for 7 days, while Drug-pSL maintained the absorbance ratio at pH 7.4 but the absorbance ratio decreased to 0.6584 at pH 6.5 and decreased rapidly at pH 5.5. Through this, it was inferred that Drug-pSL would be easily decomposed under acidic conditions, thereby releasing the compound targeting mitochondria in cancer cells.

### [Test Example 5] Evaluation of TSPO-Specific Targeting Ability of Liposome

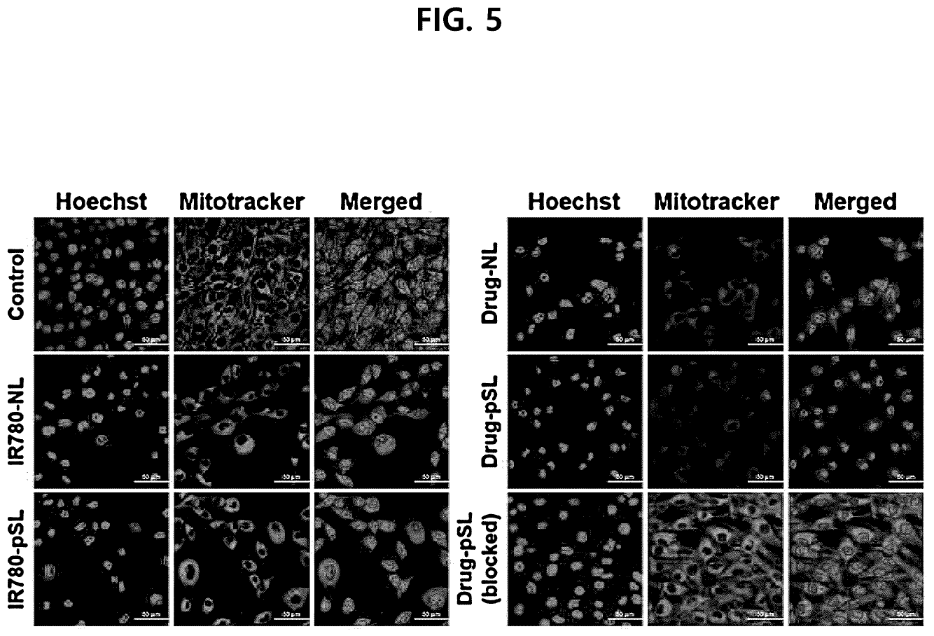

The U87MG cancer cell line was treated with each of IR780-NL, IR780-pSL, Drug-NL, and Drug-pSL at 1 μM and incubated for 24 hours. The incubated cells were stained with Hoechst 33342 and Mitotracker to image the nucleus and mitochondria. In the group in which Drug-pSL was blocked, the cells were pretreated with PK11195, known as a TSPO inhibitor, at a concentration of 100 μM for 24 hours before the cells were treated with Drug-pSL. The fluorescence signal was observed under a confocal microscope, and the change in the Mitotracker fluorescence signal is illustrated in FIG. 5.

As illustrated in FIG. 5, there was no significant difference in Mitotracker fluorescence signal in both the U87MG cells treated with IR780-NL and the U87MG cells treated with IR780-pSL. In contrast, the Mitotracker fluorescence signal decreased in the U87MG cells treated with Drug-NL and the U87MG cells treated with Drug-pSL. In the BS333 pSL blocked group that was pretreated with PK11195, the Mitotracker fluorescence signal did not decrease.

### [Test Example 6] Test of ROS Generation by Liposome

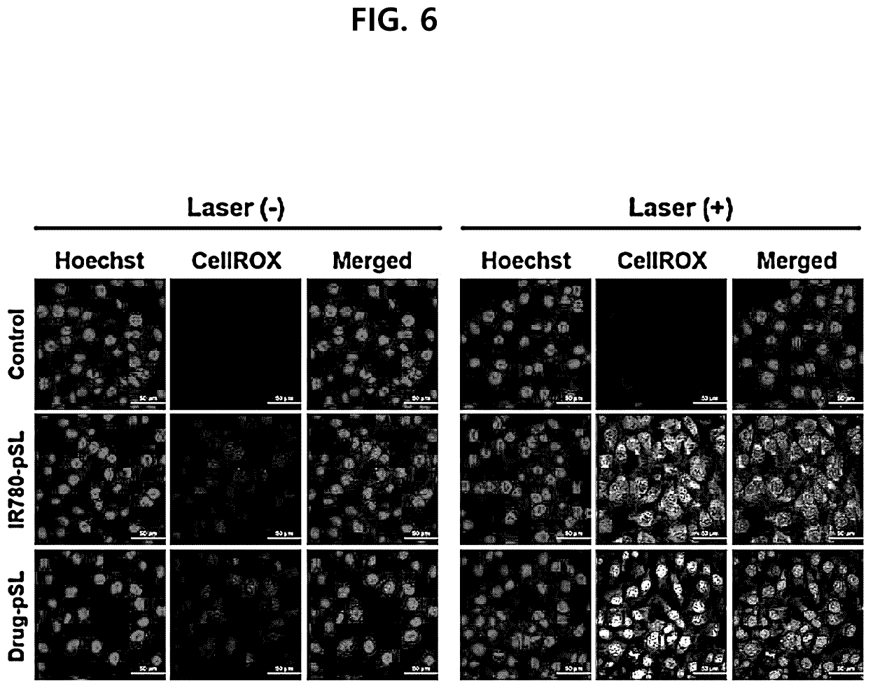

The HeLa cells were treated with each of Drug-pSL and IR780-pSN at 1 μM in a confocal dish. Next, washing with PBS was performed to remove residual liposomes, and then each sample was irradiated with an 808 nm laser at 1.5 W cm⁻² for 5 minutes. The HeLa cells irradiated with a laser were incubated at 37° C. for 2 hours in a 5% CO₂ environment. CellROX® Green Reagent (5 μM), which could detect and emit fluorescence signals from ROS, and Hoechst 33342 (10 μM) for nuclear staining were added, and then the cells were incubated for an additional 30 minutes. After washing was performed to remove the staining reagent, the fluorescence intensity of the confocal image was measured using Image J software and is illustrated in FIG. 6. The obtained fluorescence intensity was analyzed by ANOVA using Tukey's post-test and student's T test.

As illustrated in FIG. 6, strong green fluorescence was observed in the cells irradiated with a laser, confirming the generation of ROS. The fluorescence intensity in the cell nucleus was found to be higher in Drug-pSL than in IR780-pSN by 1.8 times. These results suggest that ROS, which is generated from an IR780-bound compound targeting mitochondria in cancer cells and targets TSPO, can affect not only mitochondria but also the nucleus, maximizing the cancer cell killing effect.

### [Test Example 7] Photodynamic Therapeutic Effect of Liposome In Vitro

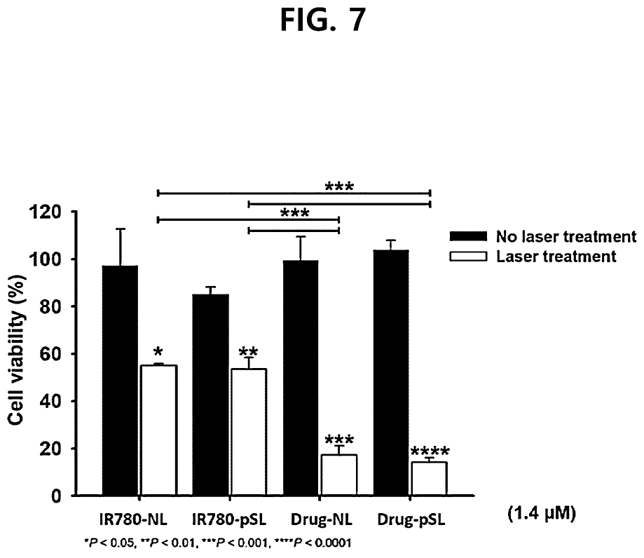

The HeLa and U87MG cancer cell lines were each treated (1×10⁴ cells well⁻¹) in a 96-well microplate and incubated at 37° C. for 24 hours under a 5% CO₂ condition. Next, the wells were treated with IR780-NL, IR780-pSL, Drug-NL and Drug-PSL in multiple concentration gradients in which the concentrations were arranged in a multiple manner so that the respective concentrations had a specific ratio difference, and the cells were incubated for 24 hours. The cell medium and liposome were removed, and then the cells were irradiated with an 808 nm laser at an intensity of 1.5 W cm⁻² for 5 minutes. The group irradiated with a laser and the group not irradiated with a laser were incubated at 37° C. for 24 hours under a 5% CO₂ condition, and the photodynamic therapeutic effect in vitro was evaluated through the MTT assay. After treatment with 0.5 mg/mL of MTT reagent, the absorbance at 540 nm was measured using a microplate reader to evaluate the cell viability, and the results are illustrated in FIG. 7.

### [Test Example 8] Photodynamic Therapeutic Effect of Liposome In Vivo

### 1. Mouse Tumor Model Containing U87MG Cancer Cell

The U87MG cancer cells were incubated, harvested in DPBS, and stored at low temperature on ice. The cells (3×10⁵ cells/10 L of PBS) were injected subcutaneously into the right thigh of each mouse.

### 2. Photodynamic Therapy (PDT) In Vivo

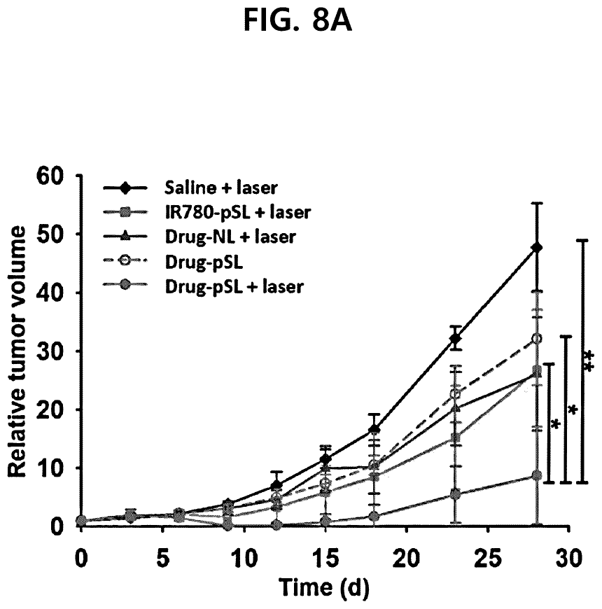

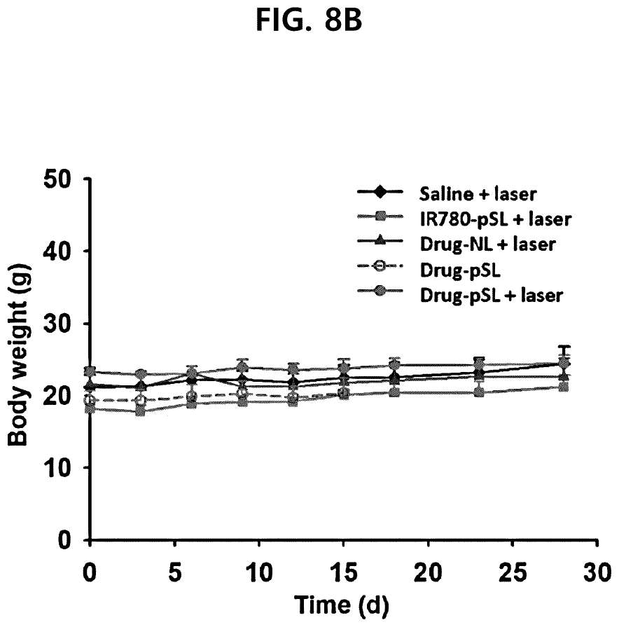

IR780-pSL, Drug-NL, Drug-pSL, and normal saline (NS) were administered to mice containing U87MG cancer cells by intravenous injection. In vivo PDT was performed by single irradiation of the tumor site with an 808 nm laser at 2 W cm⁻² for 5 minutes while the mice were anesthetized with 2% isoflurane. The tumor size and body weight of mice were measured for 28 days. The tumor volume, body weight, and survival profile were evaluated through 28 days of follow-up. The tumor volume during follow-up is illustrated in FIG. 8A, and the body weight is illustrated in FIG. 8B.

In the above, exemplary embodiments of the present invention have been described in connection with above-mentioned preferred Examples, but those skilled in the art to which the present invention pertains may make various modifications or variations based on this description without departing from the spirit and scope of the invention. Accordingly, the appended claims will include such modifications or variations as fall within the spirit of the present invention.

1. A compound represented by the following Chemical Formula 1, the compound comprising a drug and an imidazopyridine derivative: 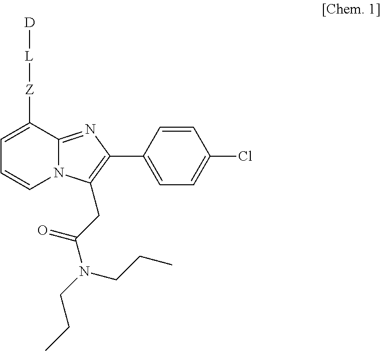
(wherein, D is a drug; L is a linker; and Z is a bonding group (conjugation)).

2. The compound according to claim 1,
wherein the drug includes any one or more selected from the group consisting of a photosensitizer, a metal chelator labeled with a metallic radioisotope, and a chemotherapeutic drug.

3. The compound according to claim 2,
wherein the photosensitizer includes one or more selected from the group consisting of porphyrin derivatives, chlorin derivatives, phthalocyanine derivatives, merocyanine derivatives, porphycene derivatives, heptamethine cyanine derivatives, chitosan derivatives, methylene blue derivatives, monoterpene derivatives, xanthene derivatives, toluidine blue derivatives, fluorescein derivatives, and menaquinone derivatives.

4. The compound according to claim 2,
wherein the metallic radioisotope includes one or more selected from the group consisting of ⁶⁰ Cu, ⁶¹Cu, ⁶²Cu, ⁶⁴Cu, ⁶⁷Cu, ⁶⁶Ga, ⁶⁷Ga, ⁶⁸Ga, ⁴⁴Sc, ⁴⁷SC, ¹¹¹In, ^{114m}In, ¹¹⁴In, ⁸⁶Y, ⁹⁰Y, ²¹²Bi, ²¹³Bi, ²¹²Pb, ²²⁵Ac, ⁸⁹Zr and ¹⁷⁷Lu, and
the metal chelator includes one or more selected from the group consisting of DOTA (1,4,7,10-tetraazacyclododecane-1,4,7,10-tetraacetic acid), DOTAGA, CB-DO2A (4,10-bis(carboxymethyl)-1,4,7,10-tetraazabicyclo[5.5.2]tetradecane), TCMC (1,4,7,10-tetrakis(carbamoylmethyl)-1,4,7,10-tetraazacyclododecane), 3p-C-DEPA, p-NH₂-Bn-Oxo-DO3A, TETA (1,4,8,11-tetraazacyclotetradecane-1,4,8,11-tetraacetic acid), CB-TE2A (4,11-bis-(carboxymethyl)-1,4,8,11-tetraazabicyclo[6.6.2]-hexadecane), Diamsar, NOTA (1,4,7-triazacyclononane-1,4,7-triacetic acid), p-SCN-Bn-NOTA (C-NOTA), NETA ({4-[2-(bis-carboxymethylamino)-ethyl]-7-carboxymethyl-[1,4,7]triazonan-1-yl}-acetic acid), TACN-TM (N,N′,N″, tris(2-mercaptoethyl)-1,4,7-triazacyclononane), DTPA (diethylenetriaminepentaacetic acid), CHX-A″-DTPA (2-(p-isothiocyanatobenzyl)-cyclohexyldiethylenetriaminepentaacetic acid), TRAP ((PRP9, TRAP-Pr), 1,4,7-triazacyclononane-1,4,7-tris[methyl(2-carboxyethyl)phosphinic acid]), AAZTA (1,4-bis(hydroxycarbonyl methyl)-6-[bis(hydroxylcarbonyl methyl)]amino-6-methyl perhydro-1,4-diazepine), H2dedpa (1,2-[[6-(carboxy)-pyridin-2-yl]-methylamino]ethane), H4octapa (N,N′-bis(6-carboxy-2-pyridylmethyl)-ethylenediamine-N,N′-diacetic acid), H2azapa (N,N′-[1-benzyl-1,2,3-triazole-4-yl]methyl-N,N′-[6-(carboxy)pyridin-2-yl]-1,2-diaminoethane), H5decapa (N,N″-[[6-(carboxy)pyridin-2-yl]methyl]-diethylenetriamine-N,N′,N″-triacetic acid), HBED (N,N′-bis(2-hydroxybenzyl)-ethylenediamine-N,N′-diacetic acid), SHBED (N,N′-bis(2-hydroxy-5-sulfobenzyl)-ethylenediamine-N,N′-diacetic acid), BPCA, CP256, PCTA (3,6,9,15-tetraazabicyclo[9.3.1]-pentadeca-1(15), 11,13-triene-3,6,9, -triacetic acid), DFO (desferrioxamine B), p-SCN-Bn-DFO, H6phospa (N,N′-(methylenephosphonate)-N,N′-[6-(methoxycarbonyl)pyridin-2-yl]-methyl-1,2-diaminoethane), HEHA (1,4,7,10,13,16-hexaazacyclohexadecane-N,N′,N″,N′″,N″ ″,N′″″-hexaacetic acid) and PEPA (1,4,7,10,13-pentaazacyclopentadecane-N,N′,N″,N′″,N″ ″-pentaacetic acid).

5. The compound according to claim 2,
wherein the chemotherapeutic drug includes one or more selected from the group consisting of doxorubicin, hydroxyurea, vincristine, docetaxel, cyclophosphamide, carboplatin, methotrexate, paclitaxel, cisplatin, 5-fluorouracil, leucovorin, prednisolone, melphalan, chlorambucil, carmustine, daunorubicin, bleomycin, cytarabine, busulfan, capecitabine, 5-FU, mitomycin C, tamoxifen, bicalutamide, gonadotropin, irinotecan, belotecan, ifosfamide, temozolomide, fludarabine, mitoxantrone, idarubicin, dexamethasone, topotecan, pemetrexed, thalidomide, gemcitabine, etoposide, letrozole, leuprorelin, azacitidine, and vinorelbine.

6. The compound according to claim 1,
wherein L is -(PEG)n-, —(CH₂)n- or -phenyl-, and
n is 0 to 20.

7. The compound according to claim 1,
wherein Z is

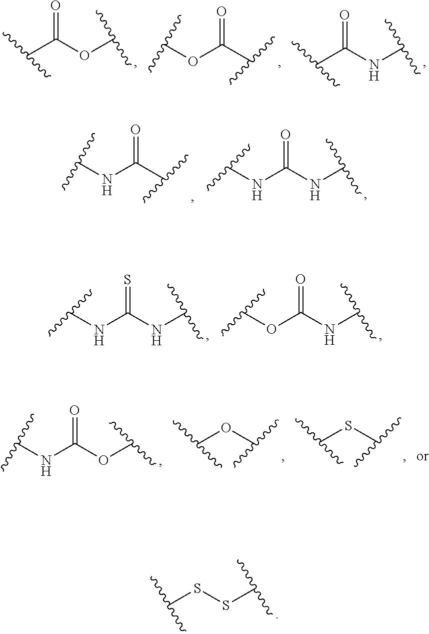

8. The compound according to claim 1,
wherein the compound is a compound represented by the following Chemical Formula 2:

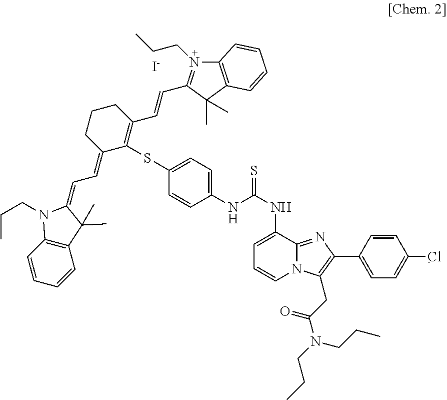

9. A liposome comprising the compound according to claim 1.

10. The liposome according to claim 9,
wherein the liposome is a pH-sensitive liposome.

11-16. (canceled)

17. A method for preventing or treating cancer, the method comprising injecting or administering a composition containing the compound according to claim 1 or a liposome comprising the compound according to claim 1 to a subject.

18. The method according to claim 17,
wherein the cancer treatment is photodynamic therapy.

19. The method according to claim 17,
wherein the cancer treatment is radiotherapy.

20. The method according to claim 17,
wherein the cancer treatment is chemotherapy.

21. The method according to claim 17,
wherein the cancer is a cancer associated with overexpression of translocator protein (TSPO).

22. The method according to claim 17,
wherein the cancer includes one or more selected from the group consisting of pancreatic cancer, lung cancer, thyroid cancer, ovarian cancer, uterine cancer, colorectal cancer, stomach cancer, bladder cancer, liver cancer, prostate cancer, breast cancer, kidney cancer and brain cancer.

23-28. (canceled)
